(19) United States
(12) Patent Application Publication
(10) Pub. No.: US 2005/0266494 A1
(43) Pub. Date: Dec. 1, 2005

(54) System and method for computer network ordering of biological testing

(76) Inventor: Timothy A. Hodge, Eads, TN (US)

Correspondence Address: BUTLER, SNOW, O'MARA, STEVENS & CANNADA PLLC, 6075 POPLAR AVENUE, SUITE 500, MEMPHIS, TN, 38119, US

(21) Appl. No.: 11/193,254

(22) Filed: Jul. 28, 2005

Related U.S. Application Data
(63) Continuation-in-part of application No. 09/945,952, filed on Sep. 4, 2001.
(63) Continuation-in-part of application No. 11/130,921, filed on May 17, 2005.
(60) Provisional application No. 60/230,371, filed on Sep. 6, 2000.

(51) Int. Cl. C12Q 1/68; G06F 17/60; G06F 19/00; G01N 33/48; G01N 33/50

(52) U.S. Cl. 435/6; 702/19; 705/3

(57) ABSTRACT

A system and method for networked ordering biological tests is provided including communicating biological test sample information and test information over the Internet.

### RELATED APPLICATIONS

This application claims priority under 35 U.S.C. s 120 as a CONTINUATION-IN-PART APPLICATION of a co-pending application entitled “System, Method and Apparatus for Transgenic and Targeted Mutagenesis Screening” which was filed on Sep. 4, 2001, and was assigned U.S. application Ser. No. 09/945,952 (the “'952 application”), and U.S. patent application Ser. No. 11/130,921 filed May 17, 2005, entitled “Systems and Methods for Ordering, Performing and Reporting Genetic Screening” the entire disclosures of which are incorporated herein by reference for all that they teach. This application and the '952 application also claim priority under 35 U.S.C. s 119(e), based on U.S. Provisional Application Ser. No. 60/230,371, filed Sep. 6, 2000, the entire disclosure of which is incorporated herein by reference for all that it teaches.

### FIELD OF THE INVENTION

This invention relates generally to biological testing. More particularly it relates to a process for ordering biological sample testing over a network such as the Internet. Even more particularly, it relates to a web-based apparatus and method for gathering information regarding an order and placing the order over the Internet.

### BACKGROUND OF THE INVENTION

Traditionally, medical, biological and biomedical research has been performed by independent researchers at research facilities. These research facilities include such institutions as hospitals, universities, colleges, pharmaceutical companies, and the like. Researchers or their research assistants take sample biological materials from cell cultures, test animals, humans, plants or other sources, prepare them, and then perform the appropriate tests upon the samples in order to determine their characteristics.

In recent years this traditional model has broken down. With the increasing costs of automated test equipment and reagents, with the increasing complexity of the test to be performed, and with the increasing accuracy required of any test results, it is more and more difficult for individual researchers to perform tests quickly and in a cost-effective manner.

Genotyping biological samples is one particular area in which it is particularly beneficial to perform accurate and precise tests quickly and in a cost-effective manner. This is true primarily because genotyping is peripheral to the actual research performed by most researchers.

“Genotyping” refers to the process of determining the biological line or strain from which a plant, animal or human is derived. In modern DNA research, much of the biological testing is performed on animals such as rats and mice that are purchased and bred to have particular genetic characteristics—i.e. particular genetic sequences in their genome. It is these genetic characteristics, and the effect of pharmaceuticals and other treatment methodologies on these animals that are of interest to researchers. Genotyping as it is described herein refers to the process of testing plant, animal, or human tissue for the existence of particular genetic sequences that define a “line” or “strain” of that plant, animal, or human. In the process described below, probes are created that are complementary to the genetic sequences that define the strain or line. These probes are placed in contact with the biological samples, typically in solution. If the genetic sequences are present in the biological samples, the probes identify their corresponding genetic sequences and the sample is said to be “positive” for those probes. If the genetic sequences are not present in the biological samples, the probes do not identify their corresponding genetic sequences and the sample is said to be “negative” for those probes. In another form of genotyping, the testing involves determining whether a plant, animal, or human has a particular genetic “profile” (instead of a “strain”) that is defined by one or more primer sets (instead of “probes”). The process described below for ordering biological testing is particularly suitable for genotyping both for strains defined by probes and for profiles defined by primer sets.

As just one example of a strain, rats and mice are often bred to have a propensity for particular cancers. This propensity is expressed in their genetic code, and biological researchers engage themselves in determining what treatment methodologies are effective for these diseases. Their research typically involves treating the animals with various carcinogenic materials, radiation, or chemical therapies to determine what triggers, what prevents, and what cures these cancers.

Nonetheless, these researchers must know for a certainty that these test animals have the cancer-causing genetic sequences for the research they conduct on these animals to be valid. Unless the animals do indeed have this propensity for cancer, any research would be in vain.

When animals are bred, however, they may or may not incorporate as part of their genome the genetic sequences of interest. As a result, even when researchers start with animals having genetic sequences of interest, they cannot always be assured that their offspring will also have the sequences, and therefore they cannot be assured that their offspring will make appropriate test subjects.

As a result, researchers typically need to perform a test on each test animal they intend to use in their research to determine whether or not each animal has a particular genetic sequence of interest—i.e. whether the animals come from the same genetic “line” of cancer-susceptibility.

While it is certainly possible for individual researchers to perform these tests, it can be expensive. The equipment needed is expensive. Furthermore, due to its cost, the some equipment is not generally available at research labs. In addition, personnel that are experienced in doing the cancer research may not be experienced in performing the genotyping necessary to screen their test animals for the cancer-susceptibility genetic sequences. Performing the tests requires constant practice in order to do it accurately and quickly which is not a trivial problem. Since the primary focus of the researchers' work is not genotyping, but research using the animals having the genetic sequences of interest, the researchers may not have the necessary practice performing the tests to ensure accurate results each time. Performing the tests is not cost-effective.

Processes have been developed for genotyping biological samples taken from test animals. A serious drawback with these processes is the need to ensure the reliability of test results and to perform the tests inexpensively. To do this, automated systems and methods for gathering data regarding the biomedical (genotyping) tests to be conducted and for ordering those tests are required. One such system and method is described in the co-pending patent applications identified herein. Since the filing of those applications, certain improvements have been made that are disclosed herein. These improvements provide for faster entry of order information, more reliable conveyance of order information to the testing laboratory, and greater ease-of-use by researchers ordering testing. This improved system of ordering biological tests (and particularly genotyping) is described and claimed herein.

### SUMMARY OF THE INVENTION

In accordance with the first embodiment of the invention, a computer-implemented method for ordering biological tests for biological samples is provided, the method comprising: electronically selecting a first strain for testing; electronically selecting a first plurality of biological samples in a first sample order to be tested for the first strain; electronically transmitting over the Internet the first strain associated with the first plurality of biological samples to a computer configured to receive orders for biological tests; and sending the first plurality of samples to a testing laboratory in a first package. The step of electronically selecting a first strain for testing may include the step of electronically selecting a second strain for testing. The step of electronically selecting a first plurality of biological samples may include the step of electronically selecting a second plurality of biological samples to be tested for the second strain. In the step of electronically transmitting may include the step of electronically transmitting over the Internet the second strain associated with the second plurality to the computer. The step of sending the first plurality may include the step of sending the second plurality of samples to the testing laboratory in the first package.

The method may include labeling the first package with a preaddressed label having first indicia before the step of sending; wherein the step of electronically transmitting the first strain includes the step of electronically transmitting the first indicia to the computer over the Internet. The preaddressed label may include at least one identifier directing the shipping company handling the first package to automatically charge shipping costs to a first entity other than the entity performing the steps above. The first entity may be the entity that tests the first plurality of samples. The method may further include the steps of: placing the first plurality of samples in a single multiwell container; and placing the single multiwell container into the first package before the step of sending the first plurality of samples.

The method may further include the steps of: placing the first and second pluralities of samples into a single multiwell container; and placing the single multiwell container into the first package before the steps of sending the first and second pluralities of samples. The method may further include the steps of: electronically creating the first strain by electronically storing a name of the first strain; and electronically storing a plurality of probes in association with the name of the first strain. The method may further include the steps of: electronically transmitting the created first strain to the computer.

The method may further include the steps of: electronically selecting a first probe from a plurality of probes; and electronically associating the first probe with the first strain. The method may further include the steps of: electronically selecting a second probe from the plurality of probes; and electronically associating the second probe with the first strain. The first strain may include a plurality of probes, and the method may further include the step of electronically deselecting at least one of the plurality of probes from the first strain.

The method may further include the steps of: electronically associating the first plurality of biological samples with the first strain for testing; and signaling the computer to automatically allocate the first plurality of biological samples to a corresponding first plurality of wells in a multiwell container in accordance with an electronically predetermined pattern of well filling; wherein the step of sending the first plurality of samples to the testing laboratory in the first package may include the step of placing the multiwell container in the package.

The method may further include the steps of: electronically associating the first plurality of biological samples with the first strain for testing; electronically associating the second plurality of biological samples with the second strain for testing after the step of electronically associating the first plurality of biological samples; and signaling the computer to automatically allocate both the first plurality of biological samples and the second plurality of biological samples to a respective first plurality of wells and a second plurality of wells in a multiwell container.

The method may further include the steps of: electronically changing the order in which the computer automatically allocates the first plurality of biological samples and the second plurality of biological samples to the first and second plurality of wells by changing the order in which the first plurality of biological samples and the second plurality of biological samples appear on a computer monitor. The step of electronically changing the order in which the computer automatically allocates the first plurality of biological samples and the second plurality of biological samples may include the step of moving a data item on the computer monitor indicative of the second plurality of biological samples upward on a sample list displayed on the computer monitor.

The method may further include the steps of: electronically associating each sample of the first plurality of samples to a corresponding location in a multiwell container; and displaying a top view of the multiwell container superimposed with first well indicia indicating each well to which a sample has been electronically associated. The method may further include the step of: displaying the top view of the multiwell container superimposed with second well indicia indicating each well to which a control sample has been electronically associated.

The method may further include the step of: displaying the top view of the multiwell container superimposed with third well indicia distinguishing wells in the multiwell container containing samples that have been individually named from wells in the multiwell container containing samples that have not been individually named. The method may further include the step of: electronically displaying a legend adjacent to the top view explaining what the first well indicia are. The method may further include the step of: individually omitting from testing any sample of the first plurality of samples that have been electronically associated to corresponding locations in the multiwell container without changing the location or order of the other samples of the first plurality of samples that have been electronically associated to corresponding locations in the multiwell container.

The method may further include the steps of: electronically associating each sample of the first plurality of samples to a corresponding location in a multiwell container; electronically associating each sample of a second plurality of samples associated with the second strain for testing to a corresponding location in the multiwell container; electronically displaying a top view of the multiwell container superimposed with fourth well indicia distinguishing well locations of the container associated with samples of the first strain, from well locations of the container associated with samples of the second strain.

The method may further include the steps of: electronically displaying a list of samples of the first plurality of samples associated with a corresponding location in the multiwell container; and electronically and automatically filling a list of names with an incremented sequence of names for the samples in the list of samples. The step of electronically and automatically filling a list of names may be based at least upon manual entry of at least two sample names in the list of names. The step of electronically and automatically filling a list of names may include the step of stopping the electronically and automatically filling a list of names when encountering any sample in the list of samples currently having a name.

The computer may be configured to block completion of an order until all of the first plurality of samples have been named, and the names have been transmitted to the computer. The method may include the step of: receiving an automated e-mail notification that the multiwell container has been received at the testing laboratory. The method may include the steps of: placing the first plurality of samples in a multiwell container having indicia; and sending the multiwell container with samples to the testing laboratory in the package; wherein the step of receiving the automated e-mail notification may include the step of receiving the automated e-mail notification generated in response to a scan of the indicia at the testing laboratory.

The method may include the step of: receiving an automated e-mail notification that biological testing of the first plurality of samples is complete. The method may include the steps of: receiving test results of the biological testing of the first plurality of samples over the Internet, the test results including an identifier of the first strain, an identifier of each probe comprising the first strain, and a list of the first plurality of samples indicating each sample by sample name and by corresponding sample results. The sample results for each sample may include an identifier indicating whether the sample was positive or negative for each probe comprising the first strain.

The test results may include an identifier of a multiwell container in which the first plurality of samples were sent to the testing laboratory. The test results may include customer account information and contact information for the testing laboratory. The test results may include a date an order for testing the first plurality of biological samples was placed, a date the first plurality of biological samples were received at the testing laboratory, and a date the biological testing of the first plurality of biological samples was completed. The test results may list each sample of the first plurality of biological samples in the first sample order. The test results may list each sample of the first plurality of biological samples in an order they were allocated to a multiwell plate.

In accordance with the second aspect of the invention, a system for ordering biological tests for biological samples is provided, including: a remote user computer comprising a CPU, and a memory comprising a RAM and a ROM wherein the computer is configured by a plurality of digital instructions to be operable to select a first strain for testing; to select a first plurality of biological samples in a first sample order to be tested for the first strain; and to transmit the first strain associated with the first plurality of biological samples to a second computer configured to receive orders for biological tests.

The remote user computer may be configured by the plurality of digital instructions to be operable to select a second strain for testing; to select a second plurality of biological samples to be tested for the second strain; to transmit over the Internet the second strain associated with the second plurality to the second computer. The remote user computer may be configured by the plurality of digital instructions to be operable to create the first strain by storing a name of the first strain entered by an operator of the remote user computer; and to store a plurality of probes for selection by the operator of the remote user computer; to associate operator-selected probes of the plurality of probes with the name of the first strain; and to transmit the operator-selected probes to the second computer in association with the name of the first strain. The remote user computer may be configured by the plurality of digital instructions to be operable to permit an operator of the remote user computer to select a first probe from a plurality of probes, and to associate the first probe with the first strain. The first strain may include a plurality of probes, and further wherein the remote user computer is configured by the plurality of digital instructions to permit the operator to first select the plurality of probes by selecting the first strain, and then to deselect at least one of the plurality of probes from the first strain. The remote user computer may be configured by the plurality of digital instructions to be operable to permit an operator of the remote user computer to associate the first plurality of biological samples with the first strain for testing; and to signal the second computer to automatically allocate the first plurality of biological samples to a corresponding first plurality of wells in a multiwell container in accordance with an electronically predetermined pattern of well filling. The remote user computer may be configured by the plurality of digital instructions to be operable to permit an operator of the remote user computer to associate the first plurality of biological samples with the first strain for testing; associate the second plurality of biological samples with the second strain for testing after the step of electronically associating the first plurality of biological samples; and to signal the second computer to automatically allocate both the first plurality of biological samples and the second plurality of biological samples to a respective first plurality of wells and a second plurality of wells in a multiwell container. The remote user computer may be configured by the plurality of digital instructions to be operable to permit an operator of the remote user computer to associate each sample of the first plurality of samples to a corresponding well location in a multiwell container, wherein the plurality of digital instructions further configure a computer display of the remote user computer to display a top view of the multiwell container superimposed with first well indicia indicating each well location to which a sample has been electronically associated. The remote user computer may be configured by the plurality of digital instructions to be operable to permit an operator of the remote user computer to associate each sample of the first plurality of samples to a corresponding location in a multiwell container and to associate each sample of a second plurality of samples associated with the second strain for testing to a corresponding location in the multiwell container; the plurality of digital instructions further configuring a computer display of said remote user computer to display a top view of the multiwell container superimposed with first well indicia distinguishing well locations of the multiwell container associated with samples of the first strain, from well locations of the multiwell container associated with samples of the second strain. The second computer may be configured to block completion of an order until all of the first plurality of samples have been named, and the names have been transmitted to the computer. The remote user computer may be configured by the plurality of digital instructions to be operable to permit an operator of the remote user computer to receive at least one of the group comprising e-mail notification that the multiwell container has been received at the testing laboratory, e-mail notification generated in response to a scan of the indicia at the testing laboratory, and e-mail notification that biological testing of the first plurality of samples is complete. The remote user computer may be configured by the plurality of digital instructions to be operable to permit an operator of the remote user computer to receive test results of the biological testing of the first plurality of samples over the Internet, the test results including an identifier of the first strain, an identifier of each probe comprising the first strain for which the first plurality of samples were tested, and a list of the first plurality of samples indicating each sample by sample name and by corresponding sample results, wherein the sample results for each sample further include an identifier indicating whether the sample was positive or negative for each probe comprising the first strain.

Numerous other features and advantages of the present invention will become readily apparent from the following detailed description, the accompanying drawings, and the appended claims, in which the like items have the same item numbers.

### BRIEF DESCRIPTION OF THE DRAWINGS

FIG. 1 is a schematic illustration of an apparatus for requesting, performing, and reporting biological testing over a distributed computer network.

FIG. 2 is a schematic illustration of the remote user computer of FIG. 1 configured to request biological testing and to receive results of that biological testing.

FIG. 3 is a schematic illustration of the web server configured to communicate with the remote user computer of FIGS. 1 and 2 to authorize an order for biological testing, to accept an order for biological testing, and to report the results of that biological testing.

FIG. 4 is a schematic illustration of the laboratory information management system (LIMS) of FIG. 1.

FIG. 5 is a picture of a package of supplies for packaging and shipping a plurality of samples from a remote user to a testing laboratory.

FIG. 6 is a chart of a process for ordering biological sample testing showing the primary web pages created during the ordering process and displayed on a screen of a remote user's computer. The illustrated web pages are generated by the web server in a browser window in response to signals are requests transmitted by a remote user to the web server.

FIGS. 7-34 illustrate the appearance of various web pages as they appear when rendered by a web browser program operating on remote user computers 102,104, on the display 280 of the remote user computers 102,104. The data displayed on these pages is either entered by the user at remote user computers 102, 104, or is generated by web server 106 in response to the operator selecting various items on the web pages. The web pages themselves are generated by web server 106.

FIG. 7 is a home page of a web site for ordering the biological sample testing.

FIG. 8 is a login page that prompts the remote user for her account name and password.

FIG. 9 is a home page of the web site that is viewable after logging into the web site.

FIG. 10 is a strained management web page for managing genetic strains for which the biological tests are conducted.

FIG. 11 is a strain creation web page for creating a new strain.

FIG. 12 is a probe creation web page for creating a new probe that defines the strain.

FIG. 13 is a probe confirmation web page confirming that a probe has been created.

FIG. 14 is a revised strain management web page illustrating a first created probe and strain.

FIG. 15 is a revised strain management web page illustrating the first created probe with two strains.

FIG. 16 is a revised strain management web page illustrating the first created probe with four strains.

FIG. 17 is a revised strain management web page superimposed with a pop-up window identifying the details of one of the probes.

FIG. 18 is a revised strain management web page illustrating two probes, a first strain defined by two probes and a second strain defined by four probes.

FIG. 19 is a strain selection web page for selecting the strains for which samples are to be tested.

FIG. 20 is a revised strain selection web page modified to permit the addition of a new strain not previously created.

FIG. 21 is a revised strain selection web page modified to display an additional well plate image, representing a second container necessary to transmit excess samples.

FIG. 22 is a sample entry web page for graphically identifying which samples have been named and have not been named, and for entering the names of samples.

FIG. 23 is the sample entry web page of FIG. 22 superimposed with a pop-up web page having data entry widgets for naming and identifying the samples for testing placed in a first well plate of FIG. 22.

FIG. 24 is the sample entry web page of FIGS. 22 and 23 superimposed with the pop-up web page of FIG. 23 scrolled to the bottom to illustrate user-selectable items for naming and identifying samples for testing placed in a second well plate of FIG. 22.

FIG. 25 is an order list web page listing all orders previously created by the user.

FIG. 26 is a revised sample entry web page showing the method of identifying named and unnamed samples in a web page image.

FIG. 27 is an order finalizing web page which summarizes the order created in the foregoing pages and permits the operator to confirm the order.

FIG. 28 is an order verification page confirming that the order illustrated on the order finalizing web page has been successfully submitted to the web server.

FIG. 29 is an account management web page illustrating basic account information stored on the web server and permitting the user to add sub-users and modify the account information.

FIG. 30 is a lower portion of the account management web page illustrating text entry widgets for modifying a shipping address.

FIG. 31 is a supply request web page for entering a request for additional supplies.

FIG. 32 is the account management web page as it would appear when generated by web server 106 to display an unregistered authorized user. A web page 3200 for creating new users is superimposed on the account management web page by web server 106.

FIG. 33 is the order web page as it is generated by web server 106 to display the order created in the above web pages after the biological tests of the order have been completed and the test results have been returned to web server 106.

FIG. 34 is the test results web page 3400

### DETAILED DESCRIPTION OF THE PREFERRED EMBODIMENTS

“Entity” as used herein refers to a natural person, a partnership, a corporation, a limited liability corporation, a limited partnership, a service corporation or a personal corporation.

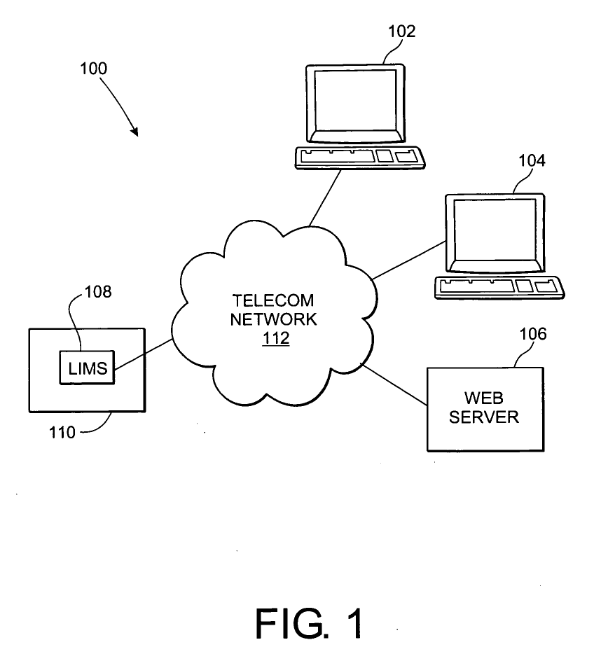

FIG. 1 illustrates an apparatus 100 for requesting, performing, and reporting biological testing over a distributed computer network. The network includes remote user computers 102, 104, a web server and order manager 106 and a computer implementing a laboratory information management system (LIMS) 108, which may be located at biological testing laboratory 110, or may be located elsewhere. Each of these is coupled together over a telecommunications network 112.

Remote user computers 102,104 may be individual personal computers that are coupled directly to the telecommunications network. They may be located at the same facility, or they may be located at different facilities. They may be owned, operated, or controlled by the same entity, or by different entities. Computers 102,104 may be joined together over a digital local area network (LAN) wherein the LAN provides access to telecommunications network 112 through the interposition of an intermediate computer or computers that are not illustrated here.

Web server and order manager (hereinafter “web server”) 106 include one or more digital computers configured to serve web pages over telecommunications network 112 to remote computers 102, 104, to gather data from the remote user at remote user computer 102,104, to create an order for biological testing, and to communicate that order to LIMS 108. Web server 106 is preferably located at a single facility. Alternatively, it may include one or more additional computers located at different facilities working in conjunction with one another.

LIMS 108 includes one or more digital computers configured with laboratory information management system (LIMS) software. It functions as a repository for data regarding samples to be tested and the test results for the samples, and may function to control automation of testing equipment (not shown in FIG. 1), to track samples, to chart workflow, and to provide electronic data capture.

Telecommunications network 112 may include public switched telephone networks (PSTN), wide area networks (WANs), and local area networks (LANS). It includes at least portions of a packet-switched public digital communication network such as the Internet.

Portions of telecommunication network 112 may include communication devices that transmit analog signals. Digital data are superimposed on these analog signals such that all communications between remote user computer's 102,104 and web server 106 are of digital data.

Biological testing laboratory 110 includes a plurality of automated digital test instruments (described, for example, in U.S. patent application Ser. Nos. 11/074,995, 09/945,952, and 11/170,477) that are configured to perform the biological testing requested by the remote user at remote user computers 102,104. The raw data from this testing is provided by the digital test instruments to LIMS 108, which in turn compiles the raw data into test results, and provides them to web server 106, which in turn provides them to the remote user at computers 102,104.

In the embodiment shown in FIG. 1, a remote user operating a remote user computer 102 or 104 places a request over the telecommunications network 112 to web server 106 requesting biological testing of a plurality of biological samples.

In response, web server 106 is configured to transmit web pages to the remote user computer that determine whether the user is authorized to place an order by requesting a previously agreed upon password and account name.

The remote user then responds via the remote user computer with the appropriate account name and password.

In response to this, web server 106 transmits a sequence of web pages that assist the remote user in identifying the particular biological samples she wishes to test.

As the remote user receives the sequence of web pages and responds as directed, web server 106 builds the user's order.

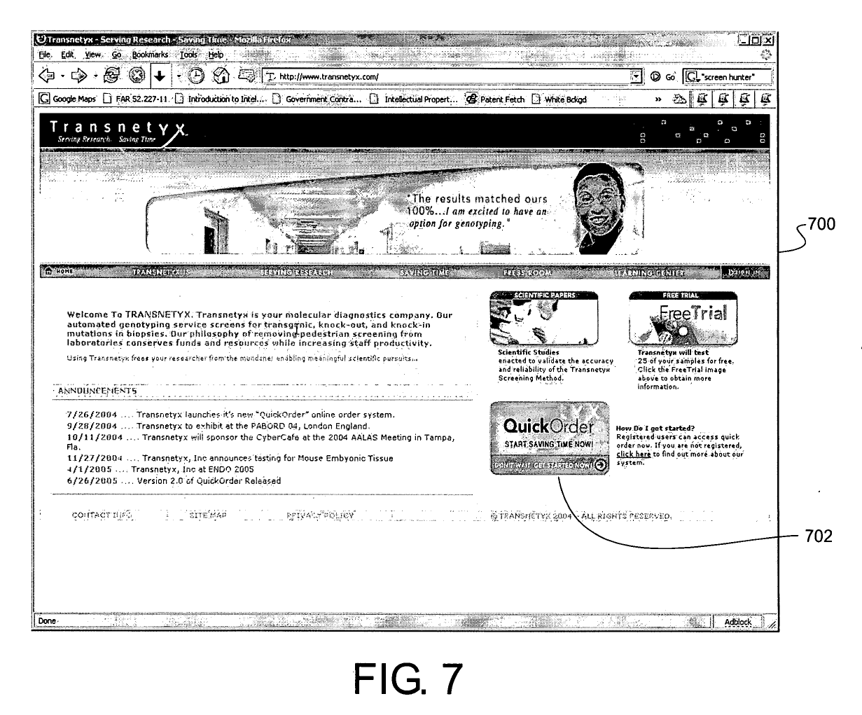

When the remote user has completed her order and signals web server 106 of that fact, web server 106 transmits the completed order to LIMS 108 for further processing. This order creation process is described herein in conjunction with FIGS. 7 to 31.

The remote user then transmits the biological samples to be tested in one or more containers identified in the ordering process to laboratory 110.

When the samples in their containers are received a laboratory 110, LIMS 108 compares the containers to identification data previously gathered by web server 106 during the ordering process from the remote user.

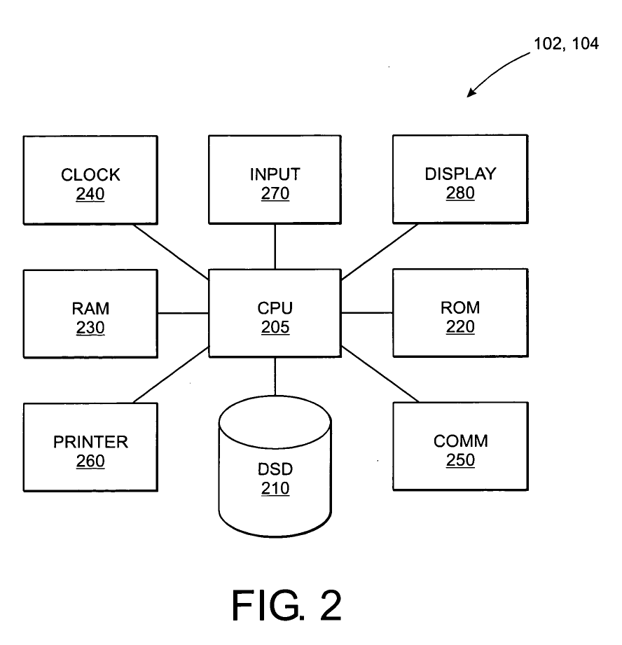

Referring now to FIG. 2, we can see a typical remote user computer 102 or 104, which comprises at least one central processing unit 205 in communication with a data storage device 210, a read-only memory (ROM) 220, a random-access memory (RAM) 230, a clock 240, a communications port 250, a printer 260, an input device 270, and a display 280.

Processor 205 is configured to be in communication with the data storage device 210, the read-only memory 220, the random access memory 230, the clock 240, the communications port 250 and the printer 260 by means of a shared data bus or, as shown in FIG. 2, dedicated connections. The input device 270 may be embodied, for example, as a keyboard, mouse, joystick or scanner or any combination thereof. The communications port 250 connects the remote user computer to the telecommunications network 112, and thence to web server 106 and LIMS 108. The communications port 250 may include multiple communication channels for simultaneous communication with more than one terminal, display 280, and/or server. The communications port 250 is configured to communicate with web server 106 by receiving web pages and transmitting responses thereto.

Data storage device 210 comprises one or more machine-readable media. Such media include, as is well-known in the art, an appropriate combination of magnetic, semiconductor and optical media, such as semiconductor memory circuits, optical disks, and magnetic disks. Storage device 210 is preferably capable of supporting storing, searching and retrieving digital data in a variety of forms, including text, image, audio, and video formats. Data storage device 210 also stores a plurality of digital instructions that configure processor 205 to communicate over communications port 250 with other computers are web servers in communication with telecommunications network 112. Among other things the plurality of digital instructions include a web browser program for browsing the Internet that is configured to interoperate with web servers such as web server 106. When operating the web browser program, processor 205 is configured to receive additional digital instructions from computers and web browsers coupled to remote user computer 102,104 by communications port 250. These instructions, received from other computers and web browsers as a part of web communications, configure remote user computer 102,104 to interoperate with these other computers and web browsers. The plurality of digital instructions may also be located in read-only memory 220 and random access memory 230.

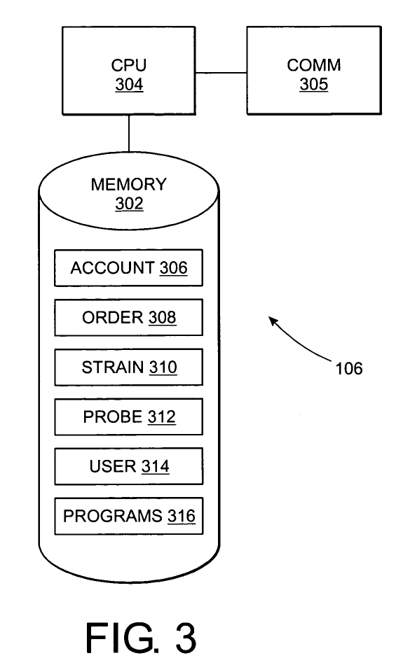

Referring to FIG. 3, a diagrammatic representation of an embodiment of web server 106 is shown. Server 106 typically includes memory 302 and at least one processor 304 in communication therewith and a communications port 305 in communication with processor 304 as well.

Memory 302 typically includes one or more machine-readable media. Such media include, as is well-known in the art, an appropriate combination of magnetic, semiconductor and optical media, such as semiconductor memory circuits, optical disks, and magnetic disks. Memory 302 is preferably capable of supporting storing, searching, and retrieving digital data in a variety of forms including text, image, audio, and video formats.

In the present embodiment, memory 302 includes an account database 306, an order database 308, a strain database 310, a probe database 312, and a user database 314.

Memory 302 also stores programs 316, which include digital instructions for controlling processor 304 in accordance with the process described herein to serve web pages. These web pages are served to the remote user computer 102, 104 to confirm the user's identity and prompt the user to enter order information necessary for creating an order.

Programs 316 also include digital instructions for controlling processor 304 to communicate with LIMS 108, to provide LIMS 108 with order information that web server 106 both receives from the remote user in the manner described herein and calculates responsive to the data received from the remote user.

Programs 316 also include digital instructions for controlling processor 304 to receive biological test results from LIMS 108 and to provide those test results to the remote user at remote user computer 102,104.

Communications port 305 may include multiple communication channels for simultaneous communication over network 112 with a plurality of remote user computers, including remote user computer 102 and remote user computer 104. Communications port 305 can communicate with the plurality of remote user computers 102, 104 by transmitting web pages to computers 102, 104 and receiving responses therefrom. Communications port 305 can also communicate with LIMS 108 to provide data received from the remote users at remote user computers 102, 104 to LIMS 108.

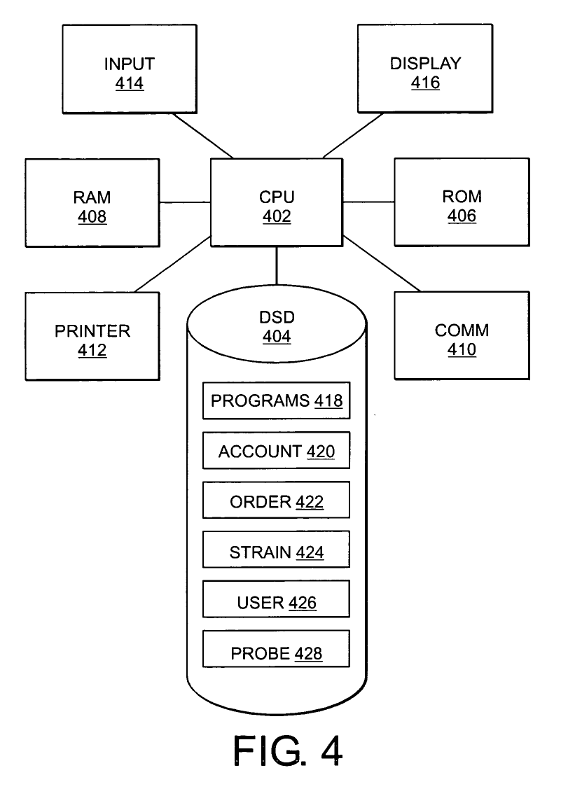

Referring to FIG. 4, a diagrammatic representation of an embodiment of LIMS 108 is shown, which comprises at least one central processing unit 402 in communication with a data storage device 404, a read-only memory (ROM) 406, a random access memory (RAM) 408, a communications port 410, a printer 412, and input device 414, and a display 416.

Processor 402 is configured to be in communication with data storage device 404, read-only memory 406, random-access memory 408, communications port 410, printer 412, input device 414, and display device 416 by means of a shared data bus or as shown in FIG. 4, dedicated connections. The input device 414 may be embodied, for example, as one or more of a keyboard, a mouse, a joystick or a scanner.

The communications port 410 connects LIMS 108 to the telecommunications network 112, and thence to web server 106 and remote user computers 102, 104. The communications port 410 may include multiple communication channels for simultaneous communication with more than one terminal, display, and/or server. The communications port 410 can communicate with web server 106 typically using a dedicated communications program, although LIMS 108 may include a Web browser.

Data storage device 404 preferably includes one or more machine-readable media. This media includes, as is well-known in the art, an appropriate combination of magnetic, semiconductor and optical media, such as semiconductor memory circuits, optical disks, and magnetic disks. Storage device 404 is preferably capable of supporting, storing, searching, and retrieving digital data in a variety of forms, including text, image, audio and video formats.

Data storage device 404 stores programs 418 which include digital instructions for controlling processor 402 in accordance with the process described herein to receive communications transmitted from web server 106 including data identifying the accounts, the users, the orders, the strains, and the probes. This data is stored in account database 420, order database 422, strain database 424, user database 426, and probe database 428. LIMS 108 receives this data from web server 106 which previously received this data from the remote user via remote user computer 102,104. The manner in which the user provides web server 106 with the data is described in conjunction with the process described in FIGS. 7-34.

The databases in web server 106 and LIMS 108 are substantial duplicates of each other. The ways in which databases update each other is well-known in the art.

Programs 418 also control processor 402 in accordance with the process described herein to communicate with the automated test equipment in biological testing laboratory 110. By doing this, processor 402 provides the test equipment with instructions indicating the type and number of samples to be tested, and the tests to be performed. Generally speaking, LIMS 108 functions as a repository for data regarding samples to be tested and the test results for the samples, to control automation of testing equipment (not shown in FIG. 1), to track samples, to chart workflow, and to provide electronic data capture. Any standard laboratory information management system software can configured to be used to provide these functions. Alternatively, a standard relational database management system such as Oracle (Oracle Corp., Redwood Shores, Calif.) or SQL Server (Microsoft Corp., Redmond, Wash.) either alone or in combination with a standard LIMS system can be used. In the preferred embodiment, the Nautilus® program (Thermo LabSystems, a business of Thermo Electron Corporation, Beverly, Mass.) is used. The manner in which LIMS 108 functions to control and monitor the process of biological testing (e.g. genomic testing), and the manner in which the testing is performed is described in more detail in co-pending U.S. patent application Ser. Nos. 11/074,995, 09/945,952, and 11/170,477.

The Ordering Process

As described above, the remote users at the remote user computers 102, 104, web server 106, LIMS 108, and operators at laboratory 110 interact to order, perform, and report biological testing. The remote users operate remote user computers 102, 104 to request that laboratory 110 perform biological testing upon a plurality of biological samples. The remote user communicates this request to web server 106. Web server 106 receives this request and the details regarding the testing to be performed on the biological samples in a communications session comprising a sequence of communications (preferably web pages and web page requests) transmitted back and forth between remote user computer 102,104 and web server 106 over telecommunications network 112.

Once the user has provided web server 106 with the minimum required order data for creating an order, web server 106 takes this order data from the remote user and transmits this order data to LIMS 108.

At substantially the same time that the remote user places the order, the user also fills a sample container or containers with a plurality of samples that are to be tested, packages and seals the containers using materials previously provided by the biological testing laboratory 110, and transmits these samples to laboratory 110, typically via a commercial shipping company (such as DHL, UPS, or FedEx).

When laboratory 110 receives the samples, the lab will have previously received an electronic record of the samples from web server 106. An operator at laboratory 110 opens the package containing the samples, electronically scans identifying data on the package into LIMS 108, and electronically scans identifying data on the container in the package holding the samples. The data on the package and the container were already transmitted from the remote user to web server 106 during the communications session between web server 106 and remote user computer 102,104.

LIMS 108 compares the two values against the values previously transmitted by the remote user to web server 106 (and thence to LIMS 108) to make sure they correspond. When LIMS 108 determines that the container identifier and the package identifier are correct, it signals web server 106 that it has received the package and container, and web server 106 is configured to automatically generate an e-mail notification that the samples were received at laboratory 110. This e-mail notification is automatically transmitted to the remote user at remote user computer 102,104.

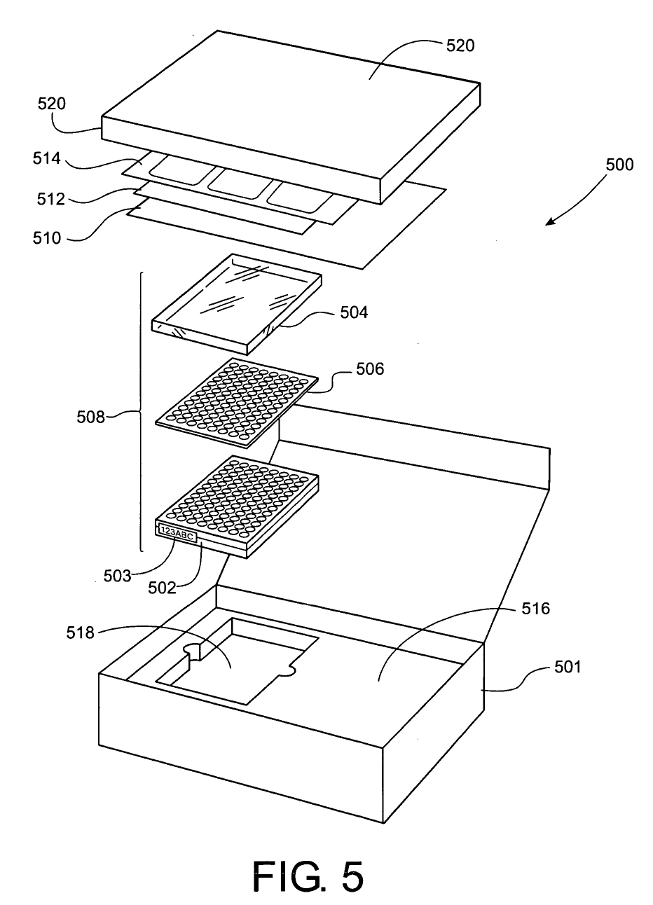

FIG. 5 illustrates components of a supply package 500 for transmitting to the remote user, typically via US mail, FedEx, DHL, or UPS that includes a box (or package) 501 enclosing an empty sample container 502, a rigid lid for the sample container 504, a cap mat 506 for fixing to the top of sample container 502 to create a sealed carrier assembly 508, a sheet of instructions 510, a mailing label 512 and several adhesive sealing strips 514 for securing sample container 502, lid 504, and cap mat 506 together for shipping. These elements are supported in box 501 in an open cell elastic foam base 516 that has a preformed recess 518 configured to receive sealed carrier assembly 508, and an open cell elastic foam cover 520.

Box 501 with its contents is sent from a supply depot (which is preferably co-located at laboratory 110) to the remote user before the user places an order for biological testing that uses container 502. As part of the ordering process, the remote user fills each well of container 502 with samples, seals the wells, places the sealed container 502 inside the box 501 and returns the box with the samples back to laboratory 110 for testing. In a preferred embodiment, box 501 as returned by the user, is not refrigerated or shipped in any specially cooled or insulated medium. Instead, it is shipped using standard package shipping systems, such a DHL, FedEx and UPS and the samples are permitted to fluctuate in temperature together with atmospheric temperature during shipment.

Before sending the box 501 back to laboratory 110, however, the remote user transmits sample and order data to web server 106 in response to the series of web pages presented by web server 106 and discussed below. In this manner, laboratory 110 has a record of box 501, container 502, and all of the samples placed in the wells of container 502 available when box 501 arrives at laboratory 110.

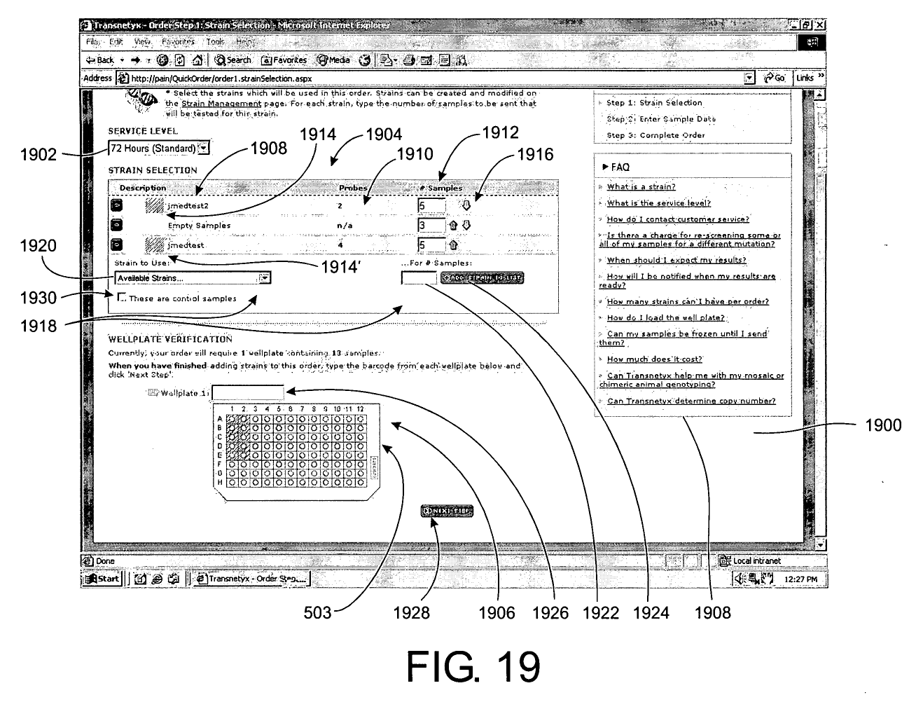

Sample container 502 is preferably a multi-well container, which defines a plurality of sample-receiving wells, preferably 96, which are preferably organized in an orthogonal array of rows and columns. In a particularly preferred embodiment, container 502 has 96 wells arranged in an 8×12 array of rows and columns. This arrangement is well-known in the art and such 96-well plates are sold by many manufacturers. Moreover, strip racks or the like may also be used. Other configurations such as linear or planar frame to which individual wells are small well assemblies may be attached also may be used. Web server 106 is configured to generate a plan view of containers 502 as well plate images 1906 and 2100, which illustrate each well of containers 502 in which the samples are placed and which illustrate which of these wells receive samples, and which of the wells have been given names by the remote user. For more detail, see the discussion below in conjunction with FIGS. 19-26.

The end of container 502 is marked with indicia 503 that uniquely identify the container 502. The indicia are typically in the form of a barcode or other electronically scannable markings. They may also be in the form of electromagnetic device such as RFID tag that can be read by a radio transmitter/receiver.

Rigid lid 504 is preferably formed as a planar sheet of plastic having flanges that extend downwardly on four sides. Lid 504 is dimensioned slightly larger than the container 500 to permit the flanges of lid 504 to extend downward around all four sides of container 502 when the lid is placed on the container.

Cap mat 506 is a flexible elastomeric sheet having a plurality of downwardly extending circular plugs that are arranged in a two-dimensional array identical in spacing and location to that of the array of wells in container 502. Each plug of mat 506 is dimensioned such that mat 506 can be placed on top of container 502 with a corresponding plug positioned over each of the plurality of wells. Each of the plugs have an outer diameter small enough to permit them to be forced into the top of and to sealed against each of the plurality of wells. In this manner, cap mat 506 can be oriented and pressed against the top surface of container 500 and to substantially simultaneously seal all the plurality of wells in container 502.

In use, the remote user fills each well of the plurality of wells with a biological sample, then presses cap mat 506 down on top of the array of well openings and seals all the wells. When this is complete, the user places rigid lid 504 on top of the cap mat 506 and seals the rigid lid to container 502 with sealing strips 514.

The user then places the sealed container 502 into recess 518 and covers the sealed container with cover 520. The user then closes lid 530 of box 501 and seals the box closed with more adhesive strips 514. The user then fixes label 512 to the outside of box 501 and contacts the shipping service to pick up the box. Label 512 is preaddressed with the address of laboratory 110 to further reduce errors. In addition, label 512 or other indicia on box 501 direct the shipping company to charge an entity associated with laboratory 110 for the shipping costs, and not to charge the remote user or the remote user's institution identified in the remote user's account information. By automatically billing a laboratory 110-related entity with the shipping costs, web server 106 can be configured to automatically add shipping charges to the costs of every order it generates as a matter of policy, thereby obviating any human interaction to work out these details during each order. This streamlines the order creation process and further decreases errors.

Electronic Order Placement

To place the order with web server 106 and laboratory 110, the remote user operates computer 102 or 104, initiating the communications session with web server 102, entering data relating to the order, and then indicating that the order, has entered, is complete and correct. Once web server 106 receives all the necessary order information, it transmits this information to LIMS 108. LIMS 108, in turn, gathers test results performed by testing laboratory 110 and returns those results to web server 106, which then forwards them to the user at remote user computer 102,104.

To remote computers 102,104 are illustrated herein to indicate that the remote user is not limited to entering all order information from a particular computer. Indeed, the remote user can save order information at one remote user computer 102 (which is thereby communicated and stored at web server 106), then, can return at a later time to communicate with web server 106 via a standard web browser on another remote user computer 104 at the same patient, or a completely different location.

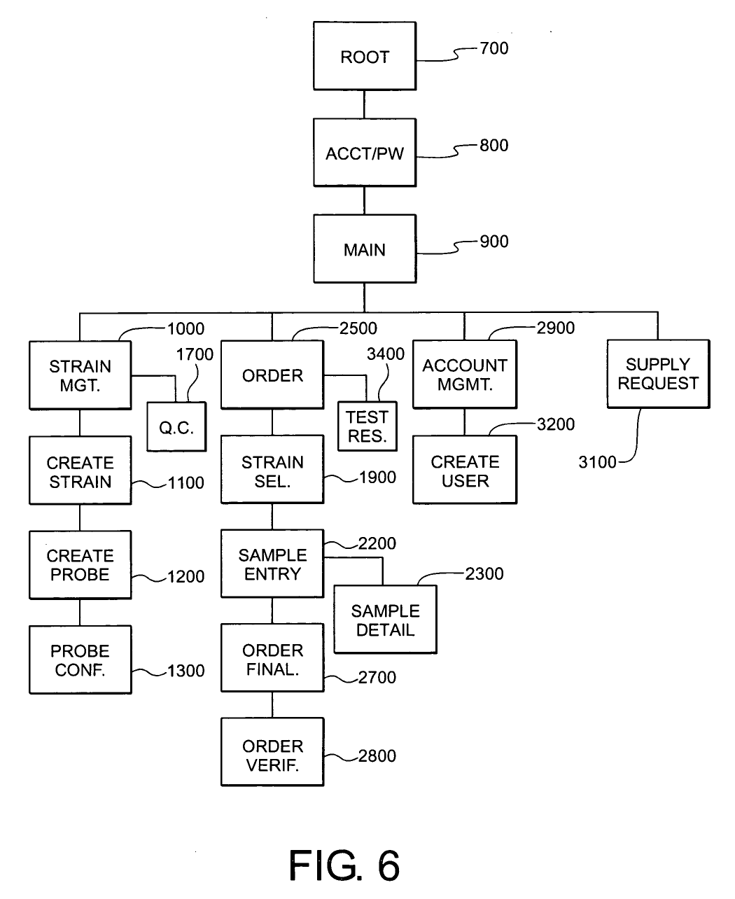

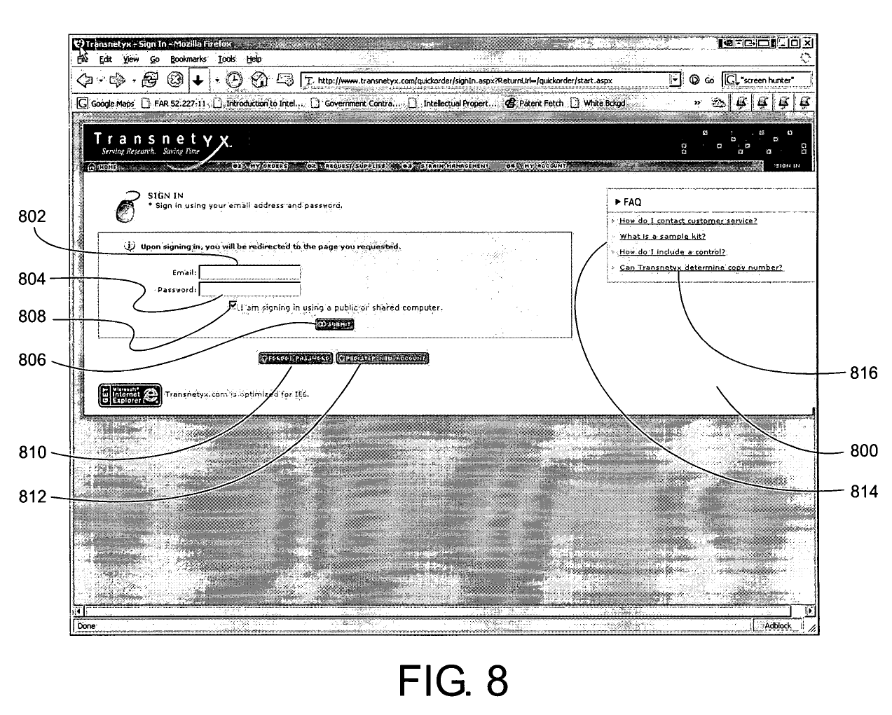

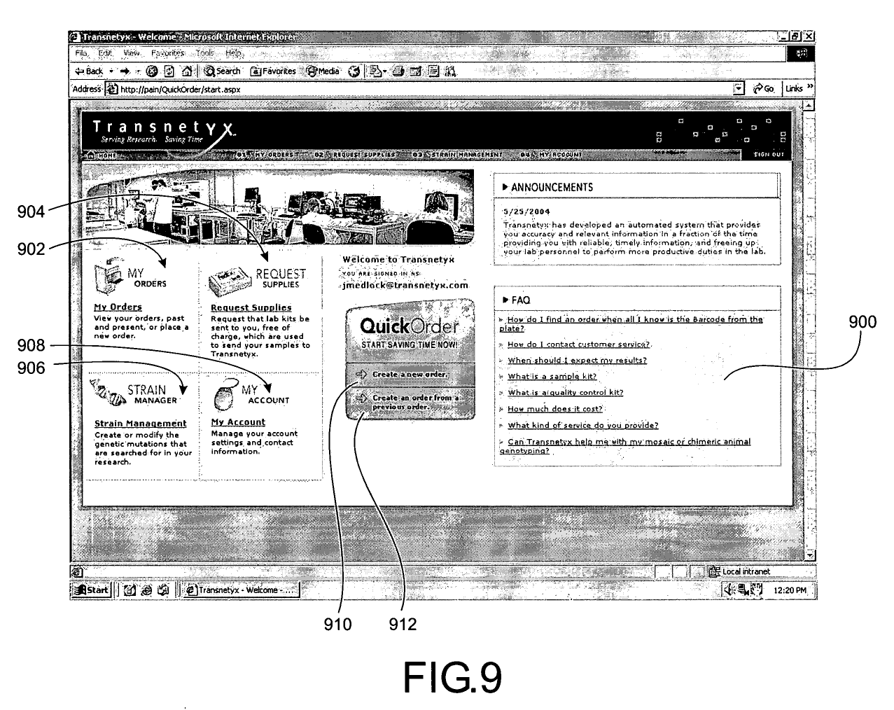

FIG. 6 illustrates the structure of web pages generated by web server 106 in response to operator manipulation of input devices 270. The first of these web pages is the root web page 700 (FIG. 7) to which the remote user first arrives. The second of these web pages is the account/password web page 800 (FIG. 8) in which the user enters her account name and password to gain access to the remaining Web pages of the web site. The third of these web pages is the main web page 900 (FIG. 9) from which the user may navigate to several other web pages.

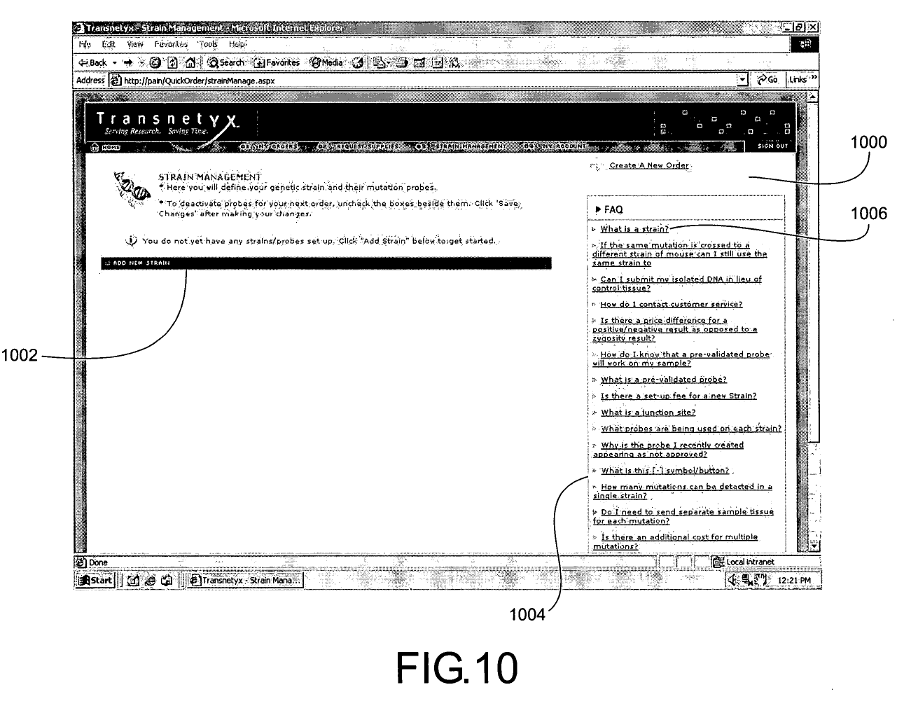

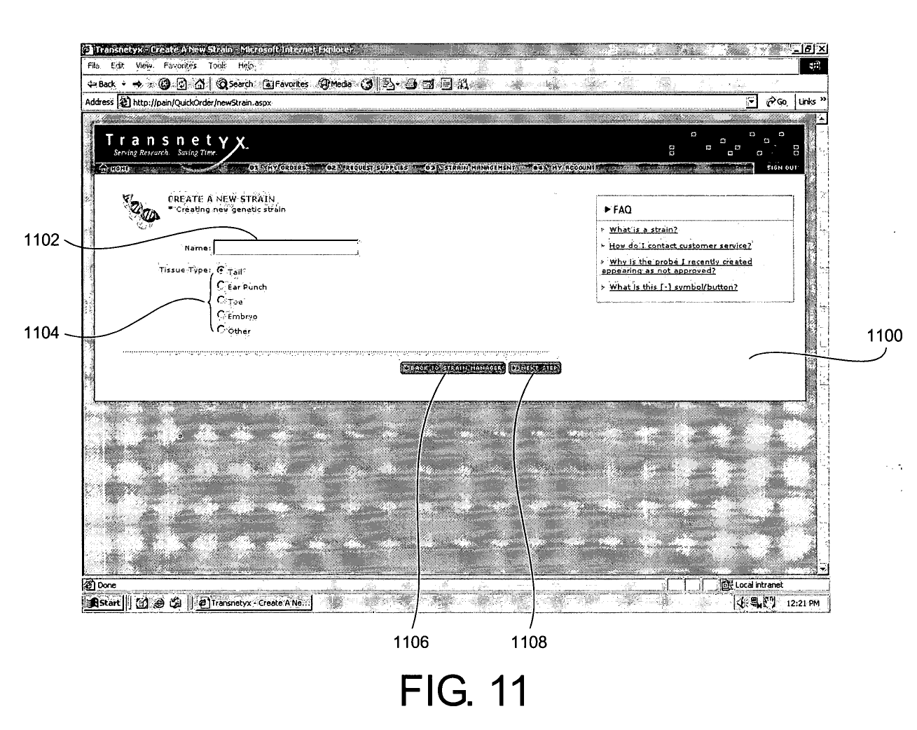

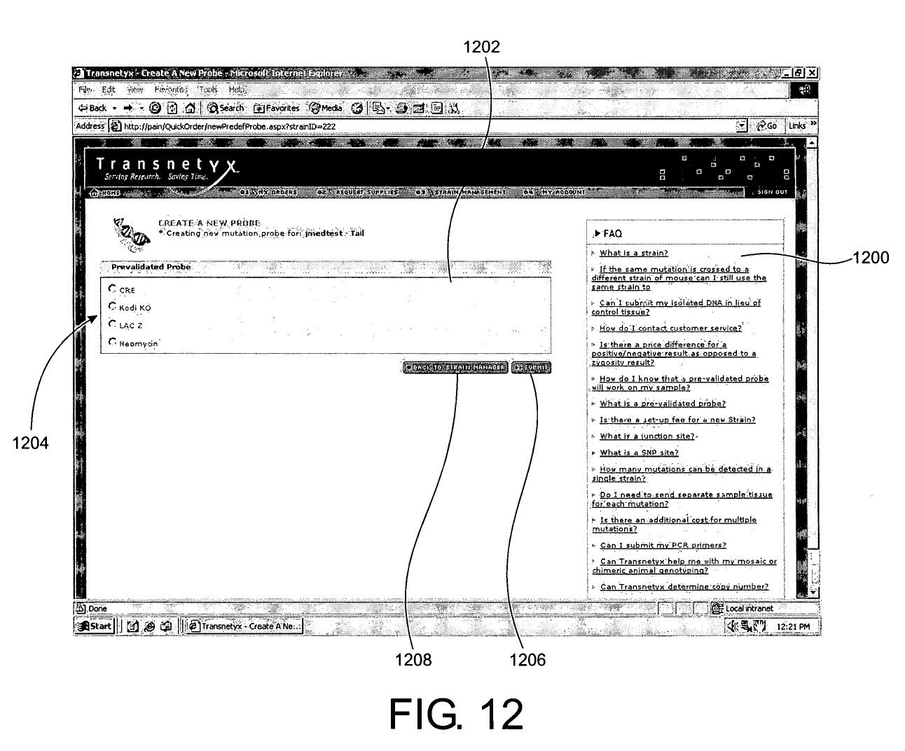

The next web page is the strain management web page 1000 (FIG. 10). In this web page, the user can view all the strains among which she can select. The next web page is the create strain web page 1100 (FIG. 11) in which the user creates a strain that she can use to test her biological samples with. The next web page is the create probe web page 1200 (FIG. 12). In this web page, the user creates probes that are then associated with a previously created strain name to define the strain.

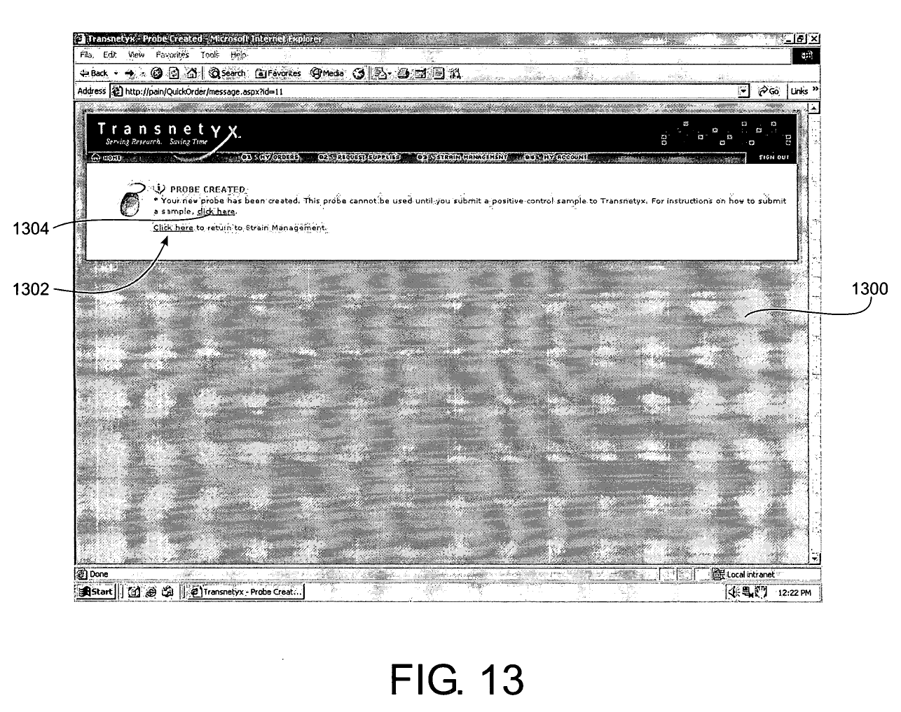

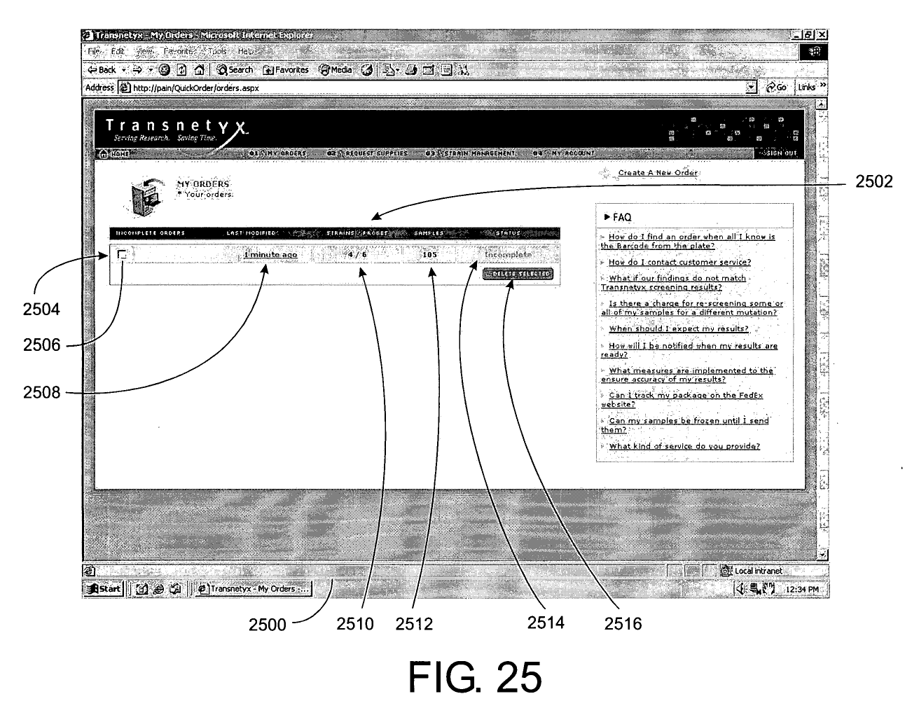

The next web page is the probe confirmation web page 1300 (FIG. 13) which confirms the user's creation of a probe. The next web page is the order web page 2500 (FIG. 25). In the order web page, the user has the opportunity to view all completed pending an incomplete orders, as well as to you test results from completed orders, to copy existing orders in order to create new ones, or to assemble an order from scratch.

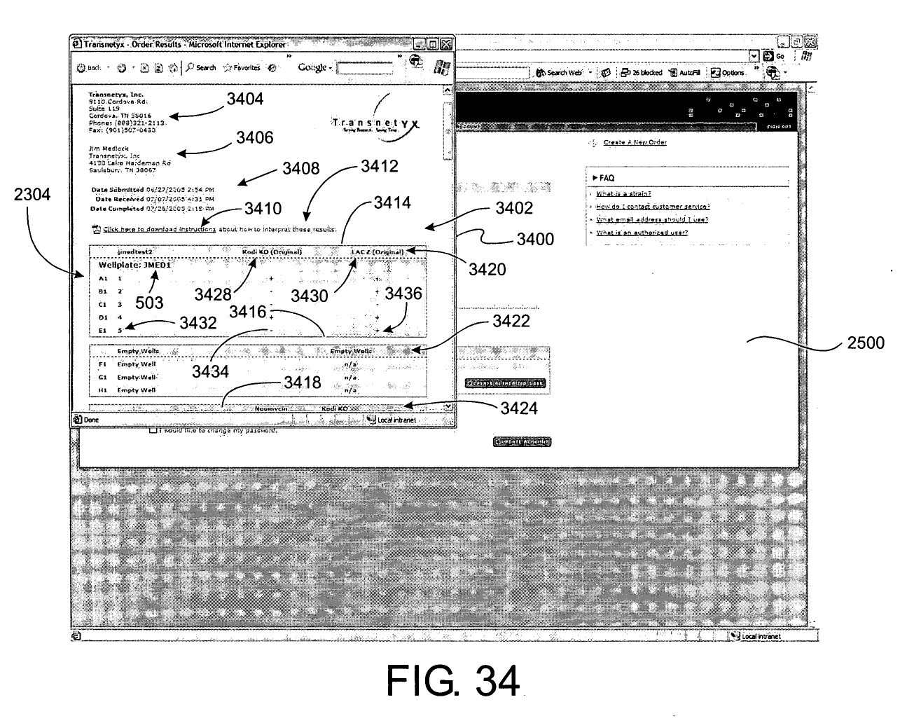

The next web page is test results web page 3400 (FIG. 34). This is the web page in which web server 106 displays the test of results from completed order. The next web page is the strain selection web page 1900 (FIG. 19). In the strain selection web page, the user selects strains for which samples are to be tested, identifies the samples that are to be tested for those strains, and allocates or associates each of the samples with a corresponding location in a container (such as a multiwell plate) in which the samples will be shipped to the testing laboratory.

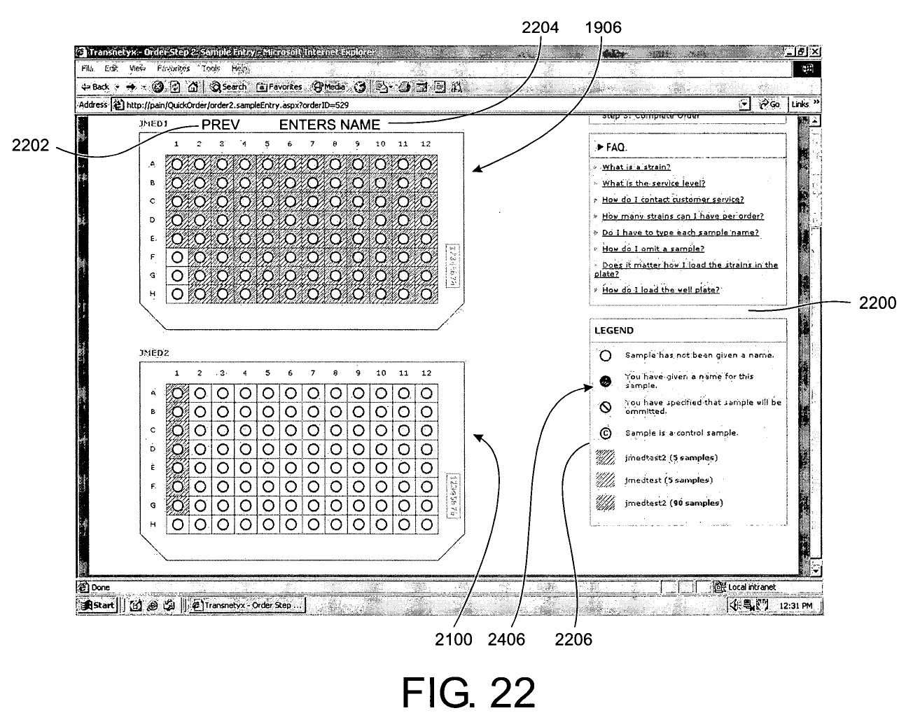

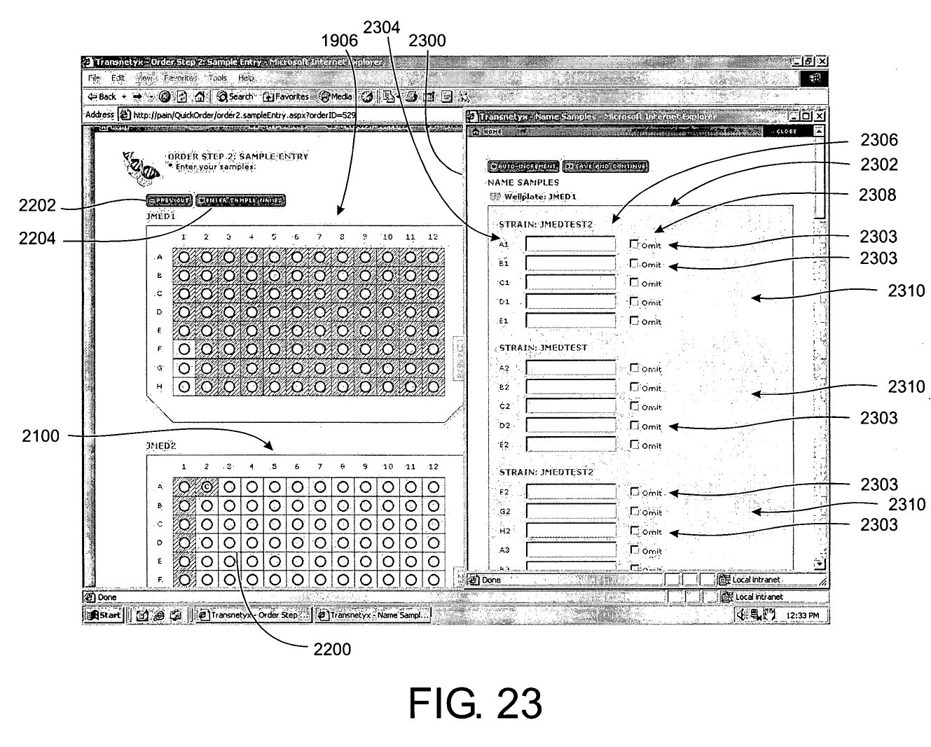

The next web page is the sample entry web page 2200 (FIG. 22). This web page displays images of the containers in which the samples will be shipped with various indicia that identify the characteristics of the tests and the biological samples to be tested. The next web page is the sample detail web page 2300 (FIG. 23). This web page displays a list of the samples that have been allocated to the container shown in the sample entry web page 2200 and in the strain selection web page 1900.

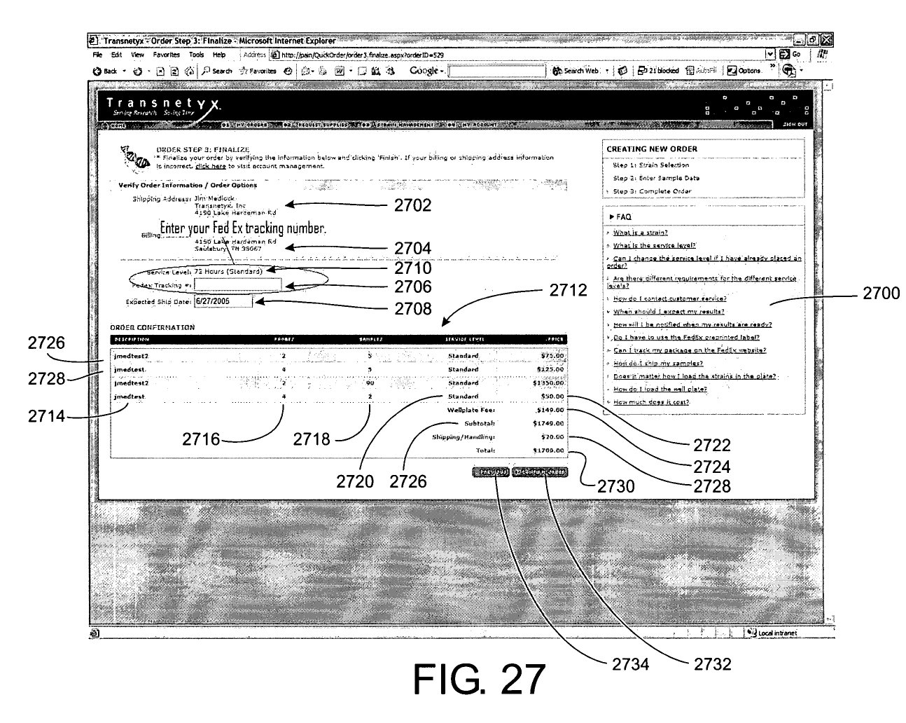

The sample detailed web page includes various items, such as text boxes and pulldown list boxes in which the user can enter additional information regarding the samples and the test to be conducted. The next web page is the order finalization web page 2700 (FIG. 27) in which the user views basic information about the order, including cost information and shipping information, before making a final approval of the order and submitting or placing the order with web server 106.

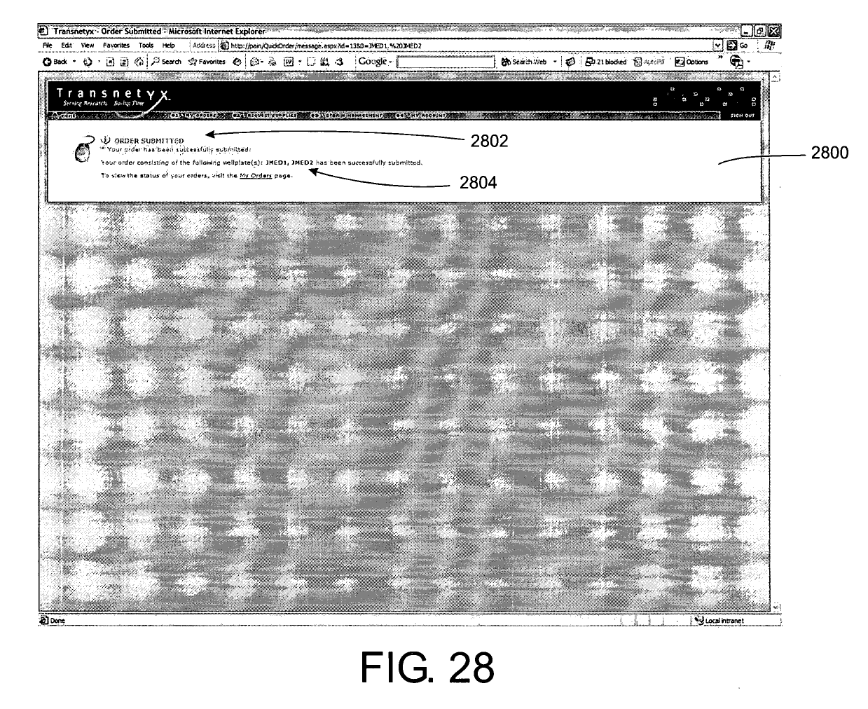

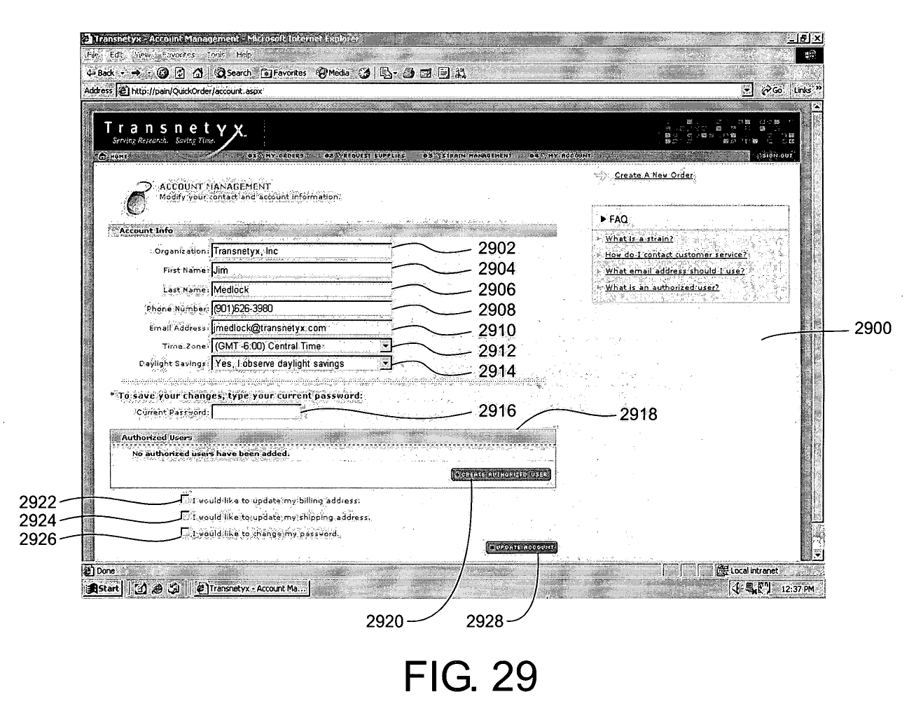

The next web page is the order verification web page 2800 (FIG. 28) which is generated by web server 106 to verify the fact that the order was placed and received by web server 106. The next web page is the account management web page 2900 (FIG. 29) which permits a master user to change basic account information, such as names, addresses, and phone numbers. It also permits a master user to authorize additional users, called “authorized users” or “sub-users” to give them access to web server 106.

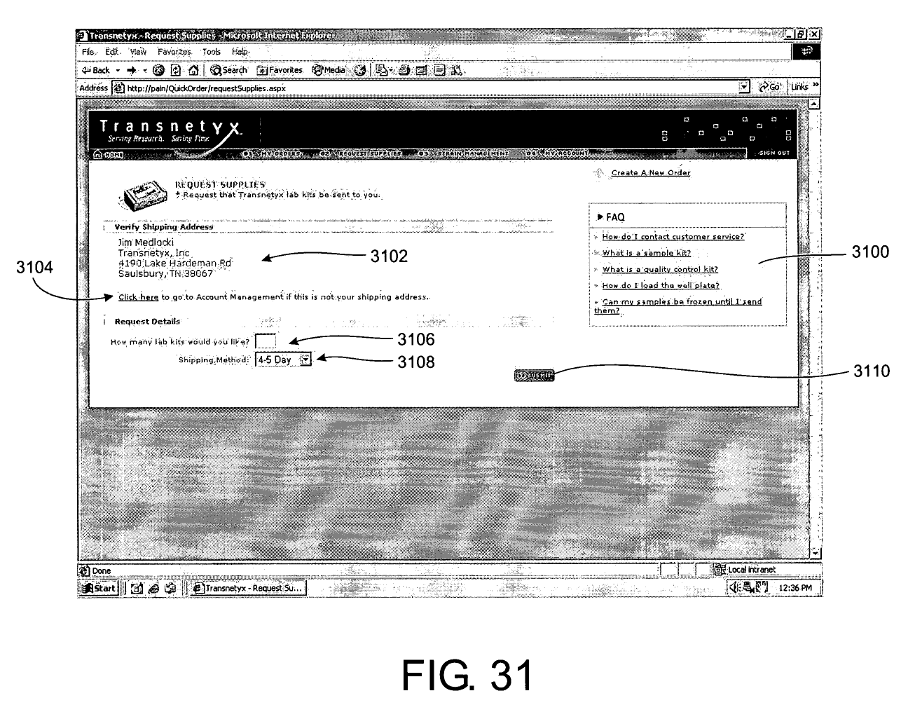

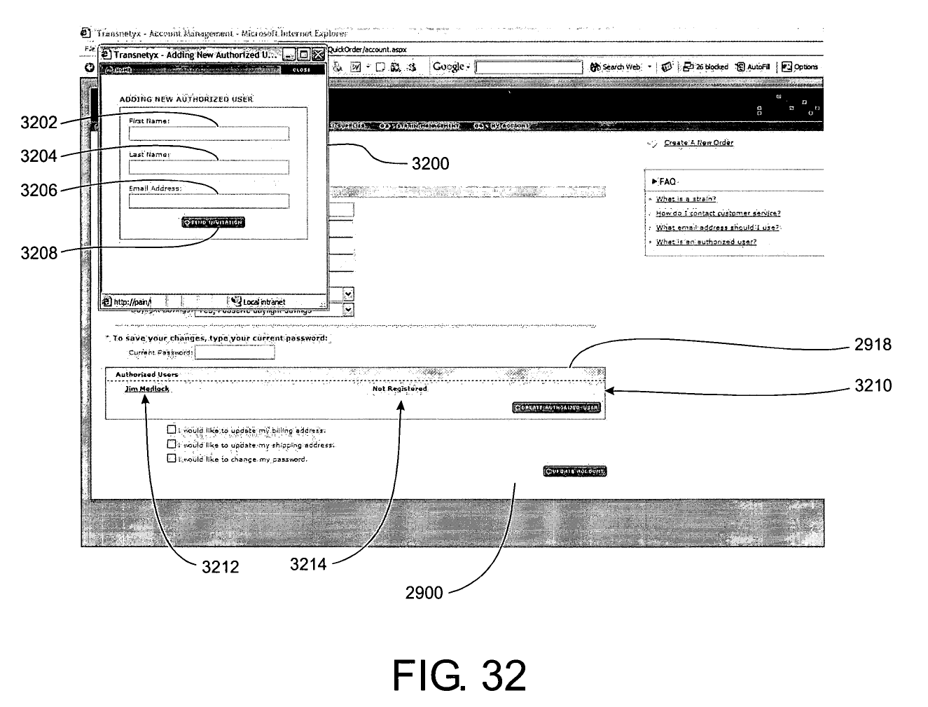

The next web page is the create user web page 3200 (FIG. 32) which is generated by web server 106 to permit the user to enter information for each new authorized user. The next web page is supply request web page 3100 (FIG. 31), in which the user can transmit a request for additional supplies to web server 106.

All of the web pages illustrated above are generated by web server 106 in response to signals received from the remote user at remote user computer 102,104. The remote user selects any of a variety of selectable items that appear on his display, such as buttons, hypertext links, check boxes, radio buttons, and the like with input devices 270 such as a mouse, keyboard, roller ball, or the like.

For ease of description, not every reference to the communication between remote user computer 102,104 and web server 106 states that the various web pages are generated (or regenerated in the same form or in a modified form) in response to signals generated by the remote user operating the remote user computer 102,104. This is the case, however. Whenever the user is described below as selecting or clicking or otherwise manipulating an item in a web page, it should be understood that a signal is transmitted from remote user computer 102, 104 to web server 106, which responsively generates or regenerates any web page that subsequently appears on display 280 of remote user computer 102,104. Check boxes, radio buttons, and text boxes are filled or selected by the remote user operating keyboards or pointing devices of input devices 270 remote user computer 102,104, and do not necessarily send a signal to web server 106.

The links illustrated in FIG. 6 between each web page show the typical paths of navigating the web site. They represent only the typical navigation paths, however. Other ways of navigating from web page to web page are described below.

The first step 600 of the process of FIG. 6 is connecting to the web server. In the step, the remote user seats herself at remote user computer 102, 104, and executes a Web browser program such as Internet Explorer, Netscape, Mozilla, or Firefox, to name just a few. Once the remote user executes a Web browser program, she enters the initial URL of the web pages served by web server 106. In a preferred embodiment, this is a root domain of a web site, for example “http:www.rootdomain.com”, wherein “rootdomain.com” refers to the root of any domain name preferred by laboratory 110.

Once the user has entered the initial URL and transmitted it to web server 106, web server 106 responds by transmitting a web page corresponding to the initial URL back to the remote user. Web browser 106 is configured to transmit each successive web page back to computer 102,104 to appear in the same window unless otherwise noted herein. Thus, the initial web page transmitted by Web browser 106 appears in the same Web browser window in which the user previously typed the initial URL.

A preferred example of this initial web page can be seen in FIG. 7. Referring to FIG. 7, the root web page 700 appears in window of a commercial Web browser. The web page includes a button 702 which the user can click using the input device of remote user computer 102,104 in order to gain access to the succeeding web pages served by Web browser 106. When the user selects button 702 (identified as “QuickOrder” in FIG. 7), web browser 106 responds by transmitting account/password entry web page 800 shown in FIG. 8.

Referring to FIG. 8, the account/password web page 800 includes a text box 802 for the user to enter an account name, here labeled as “Email:”, a text box 804 for the user to enter a corresponding password, a button 806 for submitting the data in web page 800 to web server 106, a checkbox 808 for selecting whether or not to store a browser “cookie” on the remote use computer 102,104, a button 810 for retrieving a lost password, a button 812 for registering a new account (in the event the remote user does not currently have an account), and a text region 814 for listing several frequently asked questions 816.

In response to this web page, the user enters the account identifier or name into text box 802, a corresponding password into text box 804, and clicks submit button 806. Upon receiving this data, web server 106 compares the supplied account identifier and password with the electronic records in its account database and the user database to determine whether there is a valid account associated with the remote user having that name having that password. If not, web server 106 responsively transmits an error message to the user indicating that the password and account name or incorrect. It the password and account name are valid, however, web server 106 responsively transmits the web page identified in FIG. 9.

Referring now to FIG. 9, a main web page 900 provides the user with several additional selectable choices not originally provided in the initial web page. Among these include a first button 902 that permits the remote user to view past orders, pending orders, and completed orders for biological testing, or to place a new order for biological testing, a second button 904 which permits the remote user to request supplies such as packages 500 to be used for future orders, a third button 906 that permits the remote user to create or modify the genetic mutations are genetic sequences that are searched for in the remote user's research, a fourth button 908 which permits the remote user to manage basic account information such as the remote user's name, address, telephone number, fax number, e-mail address, billing address, credit card number to be billed, and the like. Main web page 900 also includes a button 910 that permits the remote user to immediately start creating a new order, and a button 912 that permits the user to create an order using a previous order as a template. Button 912 is particularly beneficial for remote users that repeat the same biological tests for the same mutations. By using order data from a previous order, the user need only change a minimum of information, such as indicia 503 of a new container 502 that is being filled with samples.

When the operator presses button 912, a message is transmitted to web server 106 directing it to duplicate the last order made by the user. This new order is created and added to the web server's order database. Since the duplicate order has not been submitted, its status is “incomplete”, and it will be generated by web server 106 in the future in the “incomplete order” sublist of the order list discussed below. Once it appears in the order list, the remote user can select it and edit it as the user can edit any other incomplete order.

A row of buttons 914, 916, 918, and 920, labeled, respectively, “My Orders”, “Request Supplies”, “Strain Management”, “My Account”, extend across the top of main web page 900. Indeed, they extend across the top of every web page in the identical location on each web page described herein, except the web pages shown in FIGS. 7 AND 8. When the remote user clicks these buttons, they perform the same function as buttons 902, 904, 906, and 908, respectively, on main web page 900.

In the preferred embodiment, the biological tests that are performed on the samples are genotyping tests, in which the tests determined the presence or absence of a specific genetic sequence or sequences that indicate the sample was taken from a particular “line” or “strain” of animals. Hence, before any test can be conducted, the user, either alone, or in conjunction with laboratory 110, must create a strain for which the biological samples can be tested. This process is not repeated each time the remote user places in order, since the strain, once created, is saved on web server 106 in association with the remote user's account name. Once the remote user logs on to web server 106 by entering the appropriate account name and password, she is given access to all of these predefined, pre-existing strains or profiles (hereinafter collectively “strains”). As will be described below, web server 106 is configured to automatically populate selectable lists of predefined strains and the primers, probes, and primer/probe sets (hereinafter collectively referred to as “probes”) associated with those strains.

To more fully describe the operation and programming of web server 106 in conjunction with remote user computer 102, 104 we will provide an example in which a strain does not exist, and the user must create a strain before creating an order. Of course, if the strain for which testing is to be conducted has already been created, the steps of creating a strain will be unnecessary.

If the user selects button 906 (FIG. 9), thereby selecting the strain management function of web server 106, web server 106 will respond by transmitting the strain management page shown in FIG. 10 in which web server 106 lists all the strains currently existing in its strain database for the account to which the remote user has logged on.

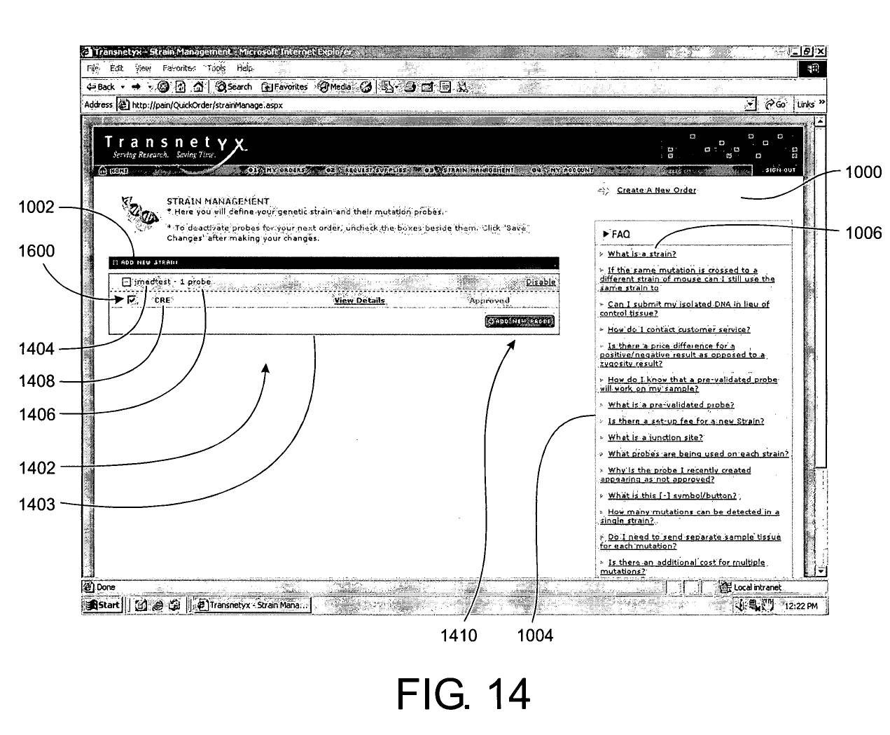

Referring now to FIG. 10, the strain management web page 1000 is illustrated. Since, in this example, no previous strain has been created, the user is presented with a button 1002 for adding a new strain. In the event a strain had been previously created and stored in the strain database of web server 106 associated with the remote user's account name, web server 106 would generate web page 1000 and populate it with a strain list of those predefined strains in its strain database. An example of a strain list with any entry is shown, for example, in FIG. 14. Since no previous strains have been created by the remote user at this point, the strain list that would otherwise appear below button 1002 is empty. Web page 1000 also includes a frequently asked questions region 1004 that includes questions 1006 relating to strains, probes, and strain management, such as, for example, the definition of “strain”, what “probes” are, what “pre-validated probes” are, and the like. Whenever the user selects any of these questions, the user is presented with an answer to the question in a pop-up window.

If the user selects button 1002, web server 106 responds by transmitting the web page shown in FIG. 11, which is the home page for creating a new strain. Any new strain needs a name, so text box 1102 is provided for the user to supply a name for the strain. In addition, a plurality of radio buttons 1104 is provided to permit the user to select the type of tissue of the biological sample to be tested. The tissue types among which the user may select include an animal's tail, ear, toe, embryo, or “other”. Web page 1100 also includes a button 1106 which permits the user to return to the previous web page 1000 without making any changes, and afford button 1108 that permits the remote user to continue to another web page (FIG. 12) corresponding to the next step in creating a strain.

Once the user has entered a desired name (in this example the name “jmedtest” was entered) and a selected the type of tissue (in this example, the tail), the remote user then clicks on the forward button 1108 and continues on to the next step in the strain creation process. In response to forward button 1108 being pressed, web server 106 transmits web page 1200 in FIG. 12 to the user. Page 1200, the home probe creation page, provides the user with a list 1202 of pre-validated probes 1204 among which the user can select. These probes include, among others, the CRE, LAC Z and Neomycin probes, well known in genetic research.

Probes are required to determine the presence of genetic sequences comprising the strain. In order to screen for particular genetic sequences (such as the ones defining the strain), those sequences must first be determined. Only when the designated genetic sequence or sequences are identified can a test to be devised to search for the existence of the sequences in the biological samples provided by the remote user to laboratory 110. There are a variety of ways that these designated genetic sequences can be acquired by the remote user or by laboratory 110. For example, if the sequences of bases that make up designated genetic sequences are known by the remote user, these sequences can be directly communicated to laboratory 110, such as to web server 106 in a web page (not shown), by e-mail, or via telephone. The remote user can indirectly communicate the designated genetic sequences to laboratory 110 by communicating the name of a publication, Journal article, gene name, sequence name, or the name of a line or strain (if the designated genetic sequence is found in animals of that line or strain), or the name of a mutation having the designated genetic sequences. Alternatively, the remote user can communicate to laboratory 110 the sequence of a primer set or probe that corresponds to a target genetic sequence of the designated genetic sequence that defines the strain or line.

In any event, whatever the genetic sequence or sequences that define the strain of animal for which the samples are tested, a probe either exists or can be created to sense the presence of those genetic sequences (or unique portions of those genetic sequences) in the samples. The process of identifying the genetic sequences that define the strain, determining the probes that can sense the genetic sequences, and validating the probe by actually testing it on biological test samples is beyond the scope of this invention, and therefore is not described in detail herein.

A detailed discussion of several such processes that can be performed by laboratory 110 based upon the data gathered herein can be found in co-pending U.S. patent application Ser. Nos. 11/074,995, 09/945,952, and 11/170,477, which are incorporated herein by reference for all that they teach.

Referring back to FIG. 12, the user selects a probe of the listed probes 1204 using the radio buttons labeled with probe names. Having selected one of the plurality of probes, the user then selects button 1206 to submit his selection to web server 106. A button 1208 is also provided, to permit the user to back out in case she does not wish to actually select a probe and add that probe to the new strain. In the event the user clicks button 1208, web server 106 responsively transmits the strain creation homepage illustrated in FIG. 11 to the user. In the event the user clicks button 1206 and thereby submits a probe selection to web server 106, the web server responsively transmits the probe confirmation web page 1300 shown in FIG. 13 to the user at remote user computer 102,104. Web page 1300 provides the user with two selections, a first button 1302 which returns the user to the strain management homepage, and a second button 1304 that provides the user with additional information regarding submitting a positive control sample to laboratory 110.

When the user selects button 1302, she signals web server 106 to re-transmit the strain management page (first shown in FIG. 10) to the remote user computer 102. This time, however, since a strain has been created (e.g. named “jmedtest”), that strain and the probe (i.e. “CRE”) just associated with the strain is listed in a strain list 1402. Strain list 1402 includes boxes 1403 (one shown in FIG. 14), one box for each strain defined by the user, that identify each strain by name 1404, by the number of probes 1406 used to identify that strain, and the name 1408 of each probe that defines the strain 1404. In the example shown in FIG. 14, only a single probe (“CRE”) is shown, since the user has associated only a single probe with the strain. However, now that the user has created a new strain by providing basic information regarding the strain (FIG. 11) and adding at least one probe (FIG. 12), the process of adding additional probes to define the strain is simplified. In the version of web page 1000 shown in FIG. 14, the user is presented not only with button 1002 to add a new strain, but with a second button 1410 (FIG. 14) to add additional probes to the definition of the strain.

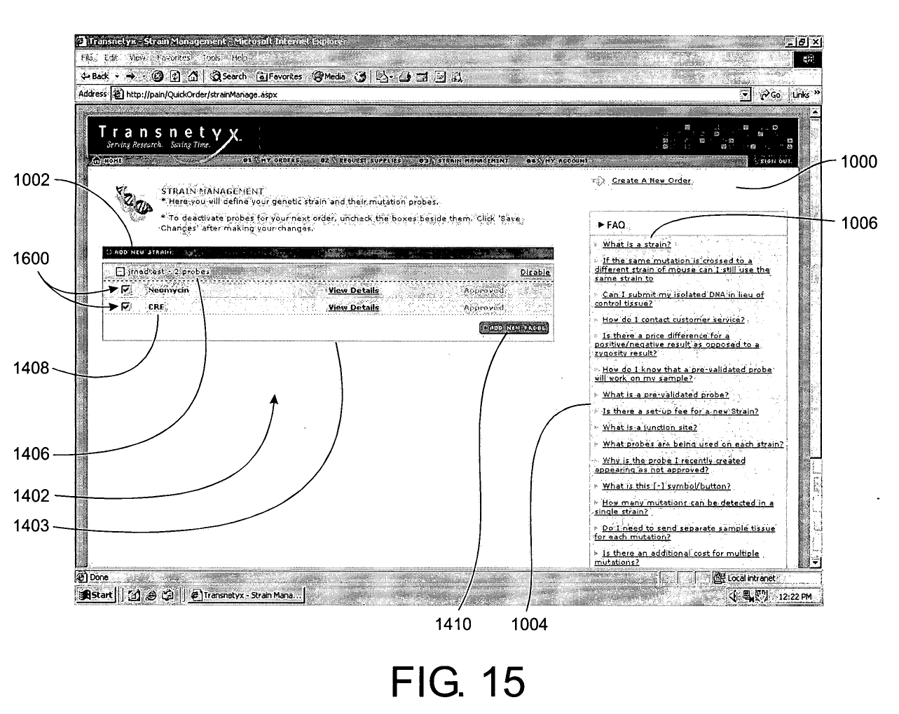

When the user selects button 1410 to add another probe to the strain, web server 106 again transmits web page 1200, listing all the pre-validated probes among which the user can select for addition to the strain. If the user selects an additional probe (e.g. “Neomycin”) and clicks submission button 1206 (FIG. 12), web server 106 responsively adds the additional probe to the definition of the strain (e.g. strain “jmedtest”) and returns a modified web page 1000 (shown in FIG. 15) in which the new probe (e.g. “Neomycin”) has been added to the list of probes 1008.

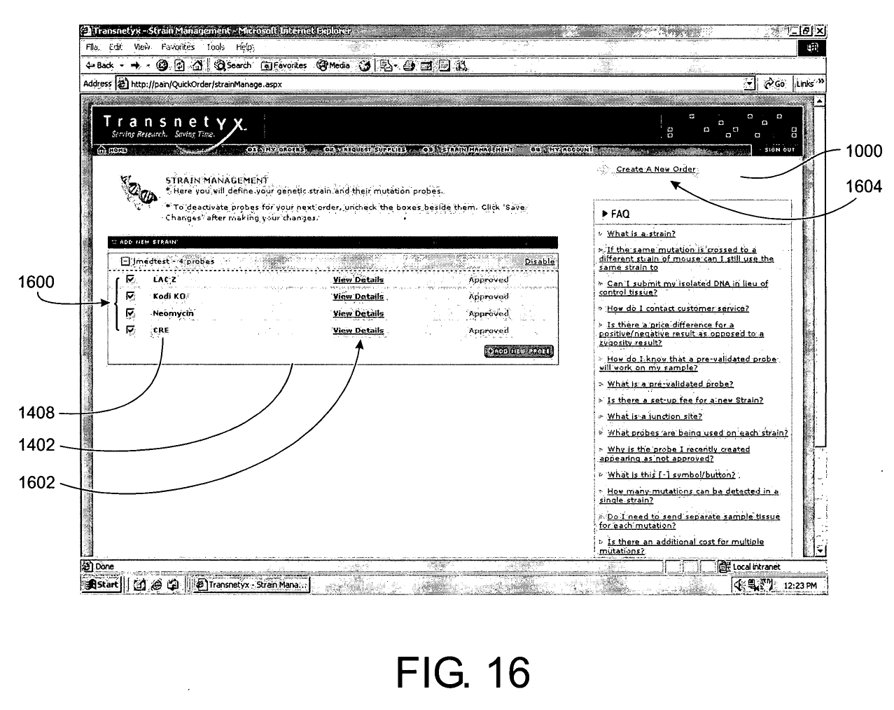

This process of adding new probes from a list of candidate pre-validated probes can be repeated as many times as necessary until the user has added sufficient probes to completely identify and define the named strain. Each time the user adds a new probe and selects the submit button, web server 106 will update the strain management page to display the new probes added to the strains. For example, consider FIG. 16, which illustrates web page 1000 after the user has added a total of four probes to the strain jmedtest” by interacting with web server 106 in the manner described above.

In this manner, web server 106 and remote user computer 102,104 have defined an item, called a “strain” herein, with a name selectable by the user that indicates genetic information. In particular, the strain name indicates one or more probes, and each of the probes indicates its own genetic sequence, which is complementary to and further indicates a complementary genetic sequence in the biological samples. Thus, the strain name, a short, easily remembered string of alphanumeric characters created by the user indicates one or more discrete genetic sequences in a biological test sample.

Likewise, the process of adding new strains by clicking button 1002 can be repeated as many times as necessary until the user has defined as many strains as she desires. Each new strain will be added below the existing strains in a box similar to box 1502, having the same buttons, but a different strain name, and the same or different probe list. Each box 1403 that identifies a strain has its own button 1410 for adding additional probes to that strain.

Typically, a researcher may identify a strain and identify several probes that are to be used to test for that strain. In some situations, a researcher may initially define that strain by several probes that, after investigation, the researcher determines are not needed. There are a variety of reasons for which this may occur. When it does, however, the researcher will typically wish to “deselect” some of the probes she previously used to test for the strain. Each probe adds an additional cost to the testing that is done, and therefore when a researcher determines that a probe is no longer necessary to identify the strain, she will typically wish to stop testing for it, and stop incurring the cost of preparing the probe.

The present system provides this capability to quickly select or deselect probes previously added to a strain simply by checking or unchecking a checkbox 1600 (FIGS. 14-16) associated with each of the probes in the definition of the strain. When checkboxes 1600 are unchecked, remote user computer 102,104 transmits a message to web server 106 indicating that, although the deselected probe is still a part of the definition of the strain, it should not be actually used at laboratory 110 when the user orders samples to be tested for that strain (e.g. “jmedtest”). Web server 106 sets a flag in the definition of the strain indicating that the deselected probe should not be used for testing. Once the user has deselected a probe in this manner, web server 106 is configured to indicate this probe should not be used with samples when the user selects the associated strain for testing.

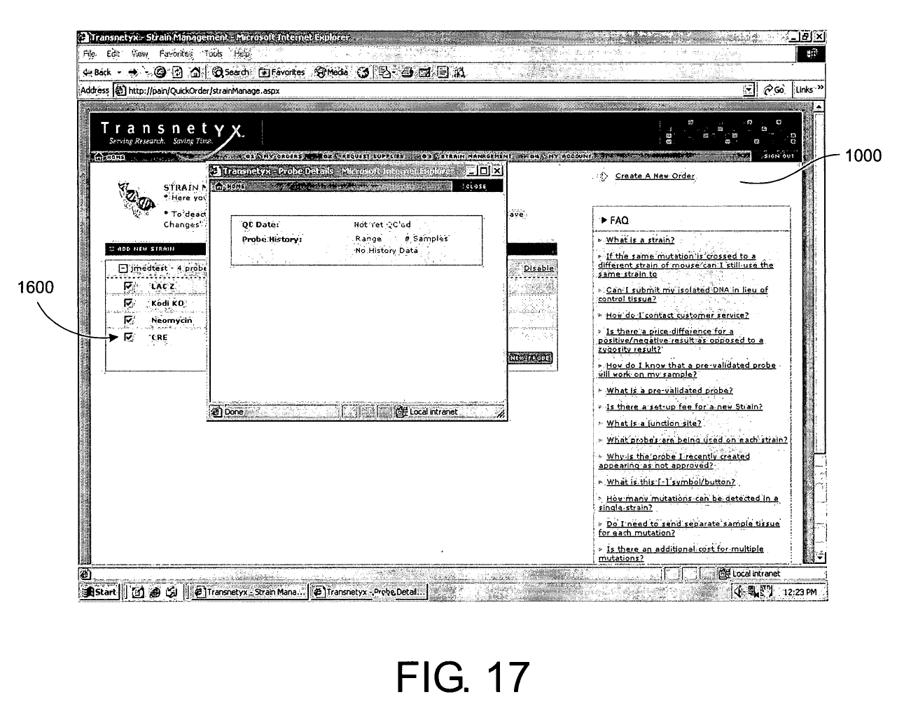

Each probe in the list of probes that the user associated with the strain includes a button 1602 labeled “View Details” on the strain management page 1000. When the user clicks on the View Details button 1602, web server 106 responsibly opens a pop-up window with web page 1700, as shown in FIG. 17. Web page 1700 is disposed on top of the window displaying the strain management web page 1000, and provides additional detail regarding the particular probe. In the example illustrated in FIG. 17, there are two fields of data that are displayed first field is the “QC Date”. There are two values possible for this field. The first value is an indicator that the probe has not yet been approved, shown in FIG. 17 as the strain “Not Yet QC'ed”. The alternative value is the date on which laboratory 110 approved the probe for use by the remote user. The second field displayed in the separate browser window indicates the range of values identified for that probe as used on samples provided by the remote user. The “approving” or “validating” of a probe, or a probe passing quality control (“QC”) refers to the probe having been used on at least two control samples provided by the remote user. Typically, the remote user will provide two biological test samples as controls, wherein one sample is known to be positive (i.e. to have the genetic sequence) and one known to be negative (i.e. to not have the genetic sequence). The remote user provides these two control samples to laboratory 110, which adds the probe to both control samples and processes them. From this result, laboratory 110 will determine a signal level corresponding to positive samples (i.e. samples having the genetic sequence) and a second signal level corresponding to negative samples (i.e. samples not having the genetic sequence). Once these two signal levels have been established, laboratory 110 can determine whether subsequent biological test samples have or do not have the genetic sequence responsive to the probe simply by looking at the level of the signal provided by the test equipment when it detects the presence of the probe. It should be clear, however, that in order for laboratory 110 to make the decision whether any sample does or does not have the genetic sequence, initial testing should be performed on a set of control samples one of which is known to have, and one of which is known to not have the particular genetic sequence to which the probe in question responds. Thus, laboratory 110 must determine the appropriate positive and negative signal levels before it can actually test sample for the genetic sequence associated with the new probe.

Signal levels can vary widely based upon, for example, the type of processing performed on the biological samples or the particular strain of animals from which the samples were taken. Thus, it is important before any probe is used on a sample from a particular remote user, to test that probe in advance. Once a signal level indicating a positive result and a signal level indicating a negative result have been determined, subsequent samples tested using the same probes can be properly categorized as either positive or negative (i.e. either having the genetic sequence are not having the genetic sequence).

On occasion, a remote user may arrange with laboratory 110 to add a probe to the list of probes displayed on the probe creation web page 1200 (FIG. 12). Laboratory 110 can add these probes to the probe database in web server 106, yet may or may not prevent them from being selected by the remote user until laboratory 110 has tested control samples with this probe and has determined corresponding positive and negative signal levels.

In the event laboratory 110 enters an unvalidated probe into the probe database of web server 106, web server 106 is configured to automatically display unvalidated probe in the probe list of web page 1200, yet to automatically display it in the probe list with an indication that it is unvalidated and therefore is not able to be designated (i.e. selected) for testing the strain for which it is listed.

When one of these unvalidated probes is selected and the remote user clicks the “submit” button 1206 (FIG. 12) thereby transmitting the unvalidated probe to web server 106, web server 106 will generate strain management page 1000 as described above, but will provide a special indication that the newly added or unvalidated probe is not available for testing any samples.

In particular, and referring now to FIG. 16, the checkbox 1600 associated with the probe will be both unchecked (to indicate it will not be used) and also grayed out (to indicate that the user cannot selected for use). The user will be able to see the probe in the probe list and therefore know that it is “in process” awaiting validation from laboratory 110, but cannot select it for testing samples.

Once laboratory 110 validates the sample and determines the appropriate signal levels to indicate a positive and negative result, laboratory personnel can edit the probe database in web server 106, changing the status of the probe in the probe database from non-validated to validated. Once this change has been made, web server 106 is configured to read the probe database and automatically display the probe in the strain management web page 1000 with the checkbox checked and in solid color (i.e. not grayed out) thereby indicating that the checkbox and probe are selectable for use in testing samples.

Once the user is identified all the strains for which she wishes to test, she must then place an order. Placing an order involves identifying all biological samples she wishes to test, the strain or strains she wishes to test the samples for, and the location of each biological sample in container 502. In addition, the remote user can transmit control samples to be tested as well.

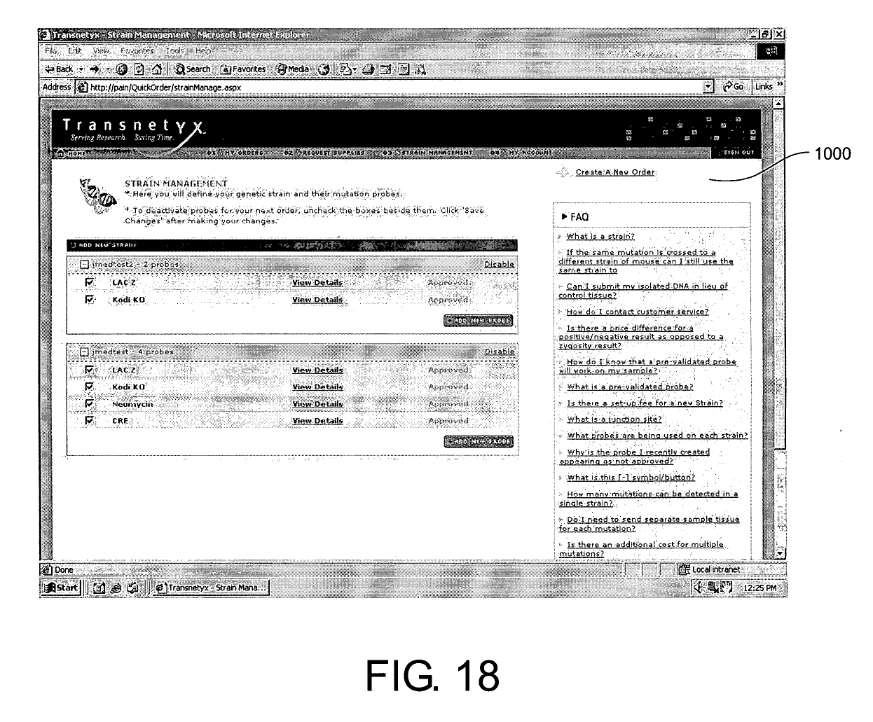

For purposes of further discussion, we will assume that the user has created to strains, identified as “jmedtest” and “jmedtest2” and shown on the strain management screen 1000 in FIG. 18. The first of these strains, “jmedtest”, is associated with four probes, LAC Z, Kodi KO, CRE, and Neomycin. The second of these strains, “jmedtest2” is associated with two probes, LAC Z, and Kodi KO.

At this point, having defined the strains by providing them with the name and identifying the probes that are to be used by laboratory 110, the remote user can simply exit the program, knowing that the strains that have been designed will be saved in web server 106, which is configured to communicate the new or amended strains to LIMS 108.

Alternatively, the user may wish to place an order for testing the biological samples, which in this context means genotyping the samples.

There are several ways the remote user may create an order. First, the strain management web page has a button 1604 the user can click to jump straight to the strain selection web pages if she is on the strain management page. Alternatively, if the user is currently on main web page 900, she can select button 910 (FIG. 9). Whenever the user selects either one of these buttons, web server 106 is configured to respond by transmitting the strain selection 1900 shown in FIG. 19.

Referring now to FIG. 19, the strain selection page 1900 includes several widgets the user can select to identify the samples she wishes to test. Having already created, either in this session, or in a previous session, at least one strain and created at least one probe associated with that strain, the user is now able to identify samples for testing, and to associate those samples with particular well locations and the strains for which they are to be tested.

FIG. 19 illustrates the strain selection page 1900, which includes a level of service selection list box 1902, a sample list 1904, and a well plate image 1906 as well as a list of frequently asked questions 1908 that relate to strain selection and ordering.

The sample list 1904 includes several lines, each line in the list including an identification (e.g. a name) 1908 for which a group of samples is to be tested, the number of probes 1910 that are both defined and activated or selected (i.e. checkbox 1600 checked) for that strain, the number of samples 1912 that are to be tested for that strain, a pattern or color icon 1914 that associate the description in the sample list 1904 with the individual wells shown in the well plate image 1906, and one or two movement icons 1916 that permit the user to move each line of samples up or down in the sample list 1904.

Just below sample list 1904 is a sample identification form 1918 which includes a drop-down list box 1920 populated by web server 106 with all of the user's strains for which the user can test samples. In the present case, this would include the two strains “jmedtest” and “jmedtest2” that were previously created by the user in the paragraphs above and shown in the strain management page 1000 of FIG. 18.

From the user's perspective, creating an order involves simply identifying the number of samples with the strain for which they are to be tested, locating them on the well plate, and giving each sample a name (which is convenient for the reporting of test results).

When the user first opens sample selection web page 1900, the sample list 1904 is empty. The sample identification form 1918 appears on the page just below the legend for sample list 1904. The legend for sample list 1904 in this embodiment includes the labels “Description”, “Probes”, and “# Samples”. The three lines or rows shown in FIG. 19 as jmedtest2”, “Empty Samples” and “jmedtest” have not been entered by the user yet. To create these rows, the user must fill out sample identification form 1918 three times in succession. To create the entries in the sample list 1904, the user first pulls down list box 1920 and selects the name of the strain for which the samples are tested (in this case “jmedtest2”). The user then enters the number of samples to be tested for that strain in the sample number text box 1922 of form 1918 (in this case 5 samples). Once these two values are entered, the user then indicates her acceptance by clicking on button 1924 of form 1918. When the user clicks button 1924, she signals web server 106 to add a new entry to the sample list 1904 and to draw a revised web page 1900 including this sample entry. At this point, the sample list 1904 of web page 1900 would only have the first line (the “jmedtest2” line) in the stain list 1904. The succeeding two lines have not been entered yet.

It must be remembered that the user must not only identify to web server 106 how many samples are to be tested for what strains, the user must also tell web server 106 where the samples are located in the container 502 which the user has filled (or will fill) with the samples she adds to the sample list. The user selects the location of the samples by selecting the order in which the samples are entered in the sample list 1904. Web server 106 is configured to automatically allocate, associate or place the samples in a particular pattern or order in the well plate images 1906 and 2100 according to a predetermined pattern determined by the programs in web server 106. Furthermore, the order in which the samples are allocated, associated or placed depends upon the location of the samples in the sample list. Web server 106 starts with the topmost group of samples of sample list 1904, and proceeds downwards to each successive group of samples until all the samples in the sample list 1904 have been allocated, associated or placed on well plate images 1906 (and additional well plate images such as well plate image 2100 if more sample locations are required to allocate, associate or place the samples than are provided in a single container 502/well plate image.

Web server 106 starts associating, allocating and placing the samples entered by the user with (i.e. allocating the samples entered by the user to) particular well locations at the upper left-hand corner of the well plate as shown in FIG. 19: well location A1. As the user designates each successive sample, web server 106 continues associating each new sample with a new well, selecting each new well for allocation in the following order: A1, B1, C1, . . . , H1, A2, B2, . . . , H2, A3, B3, . . . , H3 etc. until web server 106 has found the location for every sample identified by the user.

In the example above, the user designated 5 samples to be tested for strain “jmedtest2” and web server 106 redrew web page 1900 adding a first line to sample list 1904. This is not the only thing that web server 106 changed when it rewrote web page 1900, however. When web server 106 generated a revised web page 1900, it not only added the first line (the “jmedtest2” line) to sample list 1904, it also marked corresponding well locations A1, B1, C1, D1 and E1 in well plate image 1906 as being allocated or designated for those five samples. This allocation or designation is indicated on well plate image 1906 preferably by coloring the periphery of each of five wells with a distinctive color or pattern that matches the color or pattern of icon 1914 in the first line of sample list 1904. By using the same color or pattern, the user can, at a glance, associate one line of the strain list with the corresponding group of wells in well plate image 1906.

As the user adds successive rows to sample list 1904, web server 106 automatically creates a different color or pattern for each successive group of samples in sample list 1904.

Web server 106 is configured not only to fill in blocks of samples contiguously in the well plate image 1906, but can space groups of samples apart in well plate image 1906 by a buffer of empty wells if the remote user so designates. As shown in image 1906 of FIG. 19, a buffer or gap of three empty wells, designated F1, G1 and H1, are placed between the two groups of five samples entered by the user. The user creates a buffer of empty wells by creating a special line in sample list 1904 that is filled with a number of “samples” that are not samples, but represent empty wells that web server 106 shall designate in well plate image 1906. To do this, the user selects a special entry identified in list 1920 as “Empty Samples”. There is no strain called “Empty Samples”. Instead, when the user designates samples as “Empty Samples” and clicks button 1924, this commands web server 106 to allocate or designate a corresponding number of wells as empty wells—i.e. wells that are not associated with any sample to be tested. In this example, after identifying 5 samples to be tested for strain “jmedtest2” the user then identified 3 samples as “Empty Samples” then clicks button 1924. Web server 106 responded by calculating the location of these three empty wells and redrawing web page 1900, adding a second line to sample list 1904 named “Empty Samples”.

The user completed the process of designating samples for testing by creating a third and final line for sample list 1904, selecting a second strain (i.e. “jmedtest”), and a second number of samples (i.e. 5), and clicking button 1924. Web server 106 responded by allocating or designating five more wells in well plate image 1906 (wells A2, B2, C2, D2, E2) and creating a third line in sample list 1904.

To distinguish between adjacent samples in well plate image 1906, web server 106 is configured to automatically select a pattern (such as crosshatching) or color (such as red, orange, yellow, green, blue, indigo and violet) with which to surround each of wells A2-E2 in image 1906 that is different than the color or pattern previously applied to designated wells A1-E1. Similarly, web server 106 created icon 1914′ in the third line of sample list 1904 to have the same color or pattern as wells A2-E2 in image 1906.

For convenience, no pattern or color is applied to wells that have no samples (e.g. wells F1-H1). By maintaining the color of the empty samples the same as the color of unallocated, undesignated samples, the user is reminded that these locations are available for filling with additional samples, if necessary.

Once the user has entered all of samples she wishes to test and they appear in the sample list 1904, the user can move each group of samples (a “group of samples” corresponding to a line in sample list 1904) with respect to one another by clicking the movement icons 1916. Movement icons in the form of arrows pointing upwards move their respective lines up one row in the sample list 1904 when clicked once. Movement icons in the form of an arrow pointing downwards move their respective lines down one row in the sample list 1904 when clicked once. Successive clicks of up and down movement icons will, of course, move the lines associated with those icons in additional line up or down, respectively.

Whenever the remote user moves a line of samples up or down in the sample list 1904, web server 106 is programmed automatically move the corresponding block of allocated samples up or down in well plate image 1906.

Any of the samples identified by the user can be designated as control samples. This designation of samples as a control sample is provided by checkbox 1930 in form 1918. If, after selecting a strain, and selecting a number of samples, the user inserts a check in check box 1930, and then clicks button 1924 to add the strain to the list, Web browser 106 designates the samples as control samples, saves this information in its order database and provides this data to LIMS 108 for storage in its order database and for subsequent use by personnel at laboratory 110.

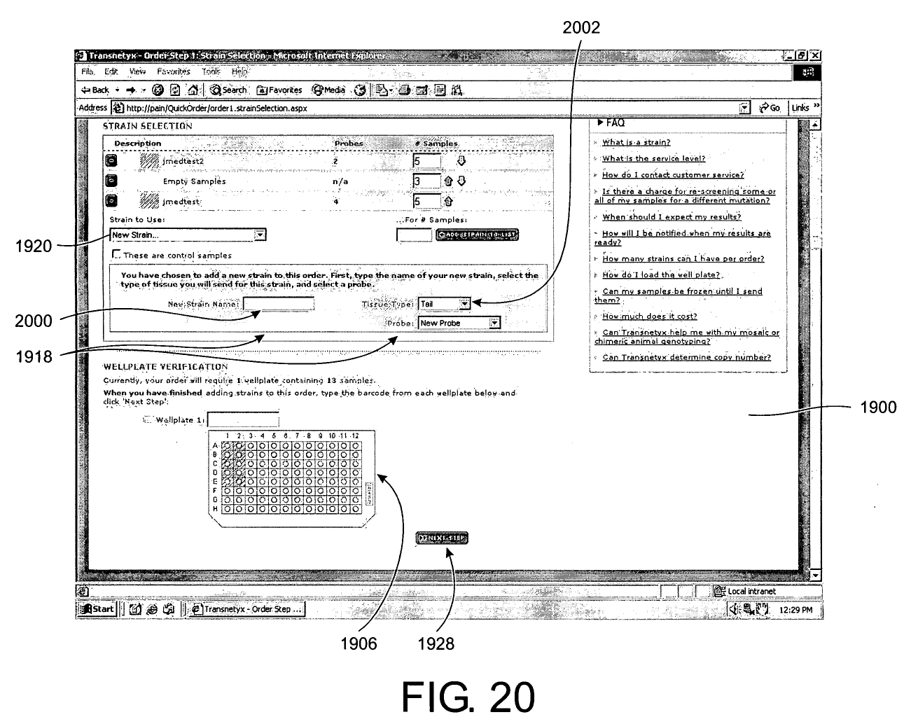

List box 1920 has an additional user selectable entry, called the “New Strain” entry. When the user pulls down list box 1920 and selects “New Strain”, Web browser 106 responds to this by generating a modified web page 1900 that has a modified form 1918, as shown in FIG. 20. Form 1918 is modified to add three additional entries: a text box 2000 for entering the name of the new strain, a drop-down list box 2002 that web server 106 fills with several different tissue types (i.e. tail tissue, ear tissue, toe tissue, embryo tissue, and other tissue) and a drop-down list box 2004 of pre-approved probes that can be used on the new strain. Web server 106 associates this information with the samples and saves it in its probe database and transmits it to LIMS 108 for storage in the LIMS 108 probe database.

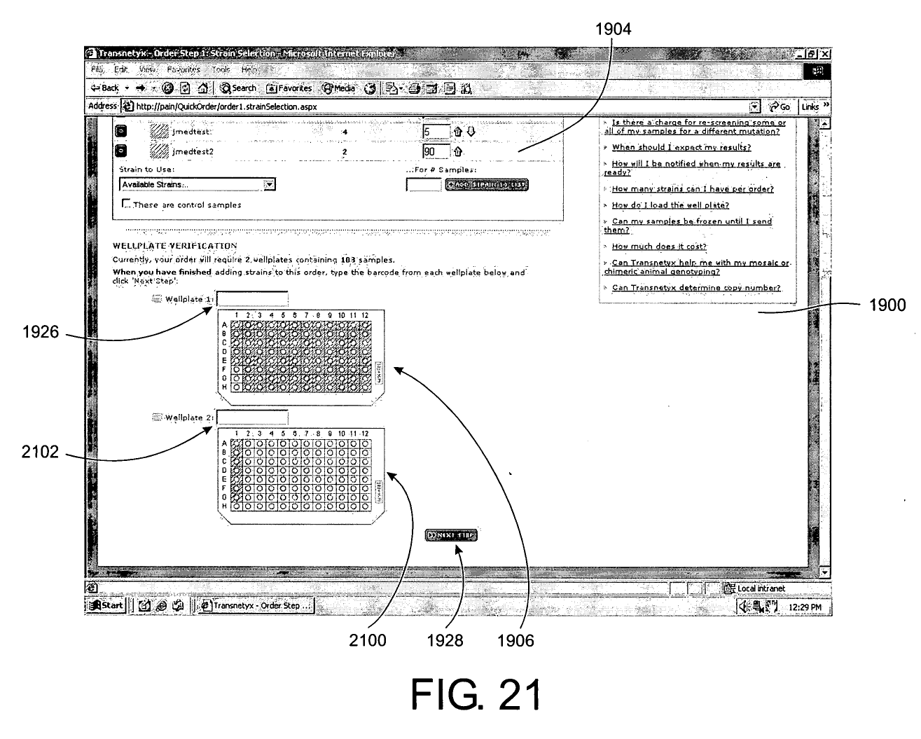

The user can designate or allocate more than 96 well locations at a time. This is illustrated in FIG. 21 which shows what happens when the user designates a further group of samples in the manner described above which are placed in sample list 1904. FIG. 21 only shows the lower portion of web page 1900 after the user has entered a fourth line in sample list 1904. The fourth line identifies a third group of 90 samples to be tested for strain “jmedtest2”. It is entered in the same manner that the previous lines are entered into sample list 1904. Note the lower portion of web page 1900. Web server 106 is configured to calculate the number of allocated or designated wells as 5 plus 3 plus 5 plus 90, for each of the lines in sample list 1904. This adds up to a total of 103 samples (including “empty samples”), which require more than one 96 well well plate to accommodate. When the number of allocated or designated samples rises above 96, Web browser 106 is configured to automatically add another well plate image 2100 to the already existing well plate image 1906 and a place this second well plate image on web page 1900. Web server 106 automatically designates or allocates all the samples into wells on the first well plate 1906, then proceeds to allocate the excess samples into wells in well plate image 2100. Web server 106 calculates that there are seven more wells needed than there are available wells on well plate 1906. Web server 106 determines that an additional container 502 will be needed, and generates an additional well plate image 2100 to which allocates the additional seven well locations. Note that Web browser 106 also adds a second text box 2102 for the user to insert the appropriate well plate identifier. As with the first well plate identifier the user type into text box 1926, the user finds the second well plate identifier 503 on a second container 502 from a second package 500 which she has at her remote facility. It is into this second container 502 that the remote user will insert the excess seven samples.

Eventually, the user will identify all of the samples she wishes to test, will identify the strain for which the samples are to be tested, and will identify the location of the samples on well plate image is 1906, 2100. At this point the user typically removes the well plate container 502 from their respective packages 500, reads the identifier off each of the container 503, and types the identifiers into text boxes 1926 and 2102. With the identifiers entered into web page 1900, the user then clicks on button 1928 and moves on to the next step in the ordering process: identifying each individual sample in all the illustrated well plates (i.e. well plate images 1906, 2100).

FIG. 22 illustrates the next step of the ordering process. In FIG. 22, sample entry web page 2200 is illustrated. This web page shows each well plate image 1906 and 2100 larger to show additional detail of each well plate and to guide the user in entering the final information. At the top of web page 2200, web server 106 displays two buttons, a first button 2020 which, when clicked, returns the user to strain selection web page 1900, and the second button 2204 that generates a pop-up window with a web page 2300 for entering each individual sample name and details of each sample. This sample detail web page 2300 is shown in FIG. 23 as it initially appears, overlaying sample entry web page 2200. Referring back to FIG. 22, sample entry web page 2200 includes a legend 2206 that identifies the initial placement of samples in the well plate images 1906, 2100. According to the legend, the image of each well identifies (1) whether a sample has been assigned to that well, (2) whether the user has named sample in that well, (3) whether the user has specified that a sample in that well will be omitted, (4) whether the user has indicated that the sample in that well is a control sample, and (5) the strain for which the sample in that well will be tested. At least initially, the plate images 1906, 2100 on sample entry web page 2200 appear the same as they last appeared in web page 1900 in all respects except size. Well plate images 1906 and 2100 on web page 2200 are larger than the same plate images on web page 1900. However, once the remote user begins entering additional information regarding the sample in sample detail web page 2300, particularly by naming each sample, web server 106 automatically regenerates web page 2200, changing the image of each well in the image to well plate 1906, 2100 to reflect the additional information added.

Initially, when sample entry web page 2200 is displayed, the user has an opportunity to review the current well assignments and to make any changes necessary by clicking on button 2202, returning to web page 1900, and moving items in sample list 1904 up and down with movement buttons 1916 or by adding or removing samples (FIG. 19).

The remote user can also review data associated with any of the well locations in well plate images 1906, 2100, merely by moving a screen pointer, such as a cursor directed by mouse or trackball, across the well location in question. Web page 2200 includes data regarding each of the well locations, including the strain name and the sample name (when the remote user names that sample). Each time web page 2200 is regenerated by Web server 106, web server 106 embeds new information in web page 2200 for any new samples the user has named in web page 2300.

Whenever the user moves a screen pointer across a well location in well plate images 1906, 2100, the browser on remote user computer 102,104 is configured to display a small data box adjacent to that well location listing the strain name and the name of the sample that web server 106 expects the remote user to place in the corresponding well.

Assuming the remote user is satisfied with the present location number and type of each sample to be tested and shown in well plate images 1906, 2100, the remote user then presses button 2204 signaling web server 106 to open sample detail web page 2300. In web page 2300, web server 106 displays a list 2302 of all samples identified in sample list 1904 and shown as allocated to a well in well plate images 1906, 2100. List 2302 does not display every well location in the well plate images, since not every well location is allocated to a sample. Web server 106 does not list well locations F1, G1 and H1, which in the previous example were identified by the user as being “Empty Samples” (wells intended never to be filled with a sample).

Each line 2303 in the list 2302 includes a well location identifier (A1, A2, etc.) 2304, that indicates the physical location of the well on the well plate images, a text box 2306 in which the remote user can enter an alphanumeric named for the sample in that well, and a check box 2308 that, when checked, directs web server 106 to omit the sample from testing.

Regarding the last item, check box 2308, upon occasion the user may make a mistake with samples when placing them in a container 502. When this happens, the remote user is not required to dispose of the container 502 and its samples and refill a new one, or to keep the existing container 502 and pay for an unnecessary test on a mistaken sample. Instead, the remote user is given a chance to indicate any of the wells as not to be tested or to be “omitted” from testing as indicated by the legend next to check box 2308. This adds an additional efficiency to the sample ordering process and accommodates human error.

The lines in the list 2302 are divided into groups of samples 2310, each group 2310 corresponding to one of the lines in sample list 1904 (FIG. 19). Thus, just as sample list 1904 has three lines of samples, a first line with five samples testing for strain “jmedtest2”, a second line with five samples testing for strain “jmedtest”, and a third line with 90 samples testing for strain “jmedtest2”, so list 2302 has three subgroups of samples, listed in the same order, group as five samples of “jmedtest2”, five samples of “jmedtest”, and a final 90 samples of “jmedtest2”.

At this stage, the user must, at a minimum, provide a name for each of the samples. To facilitate this process, web server 106 is configured to examine the names of samples entered into text boxes 2306 by the user, to identify a periodically incrementing or decrementing pattern in those names, to generate succeeding names matching that pattern, and to automatically fill the remaining text boxes 2306 with those generated names.

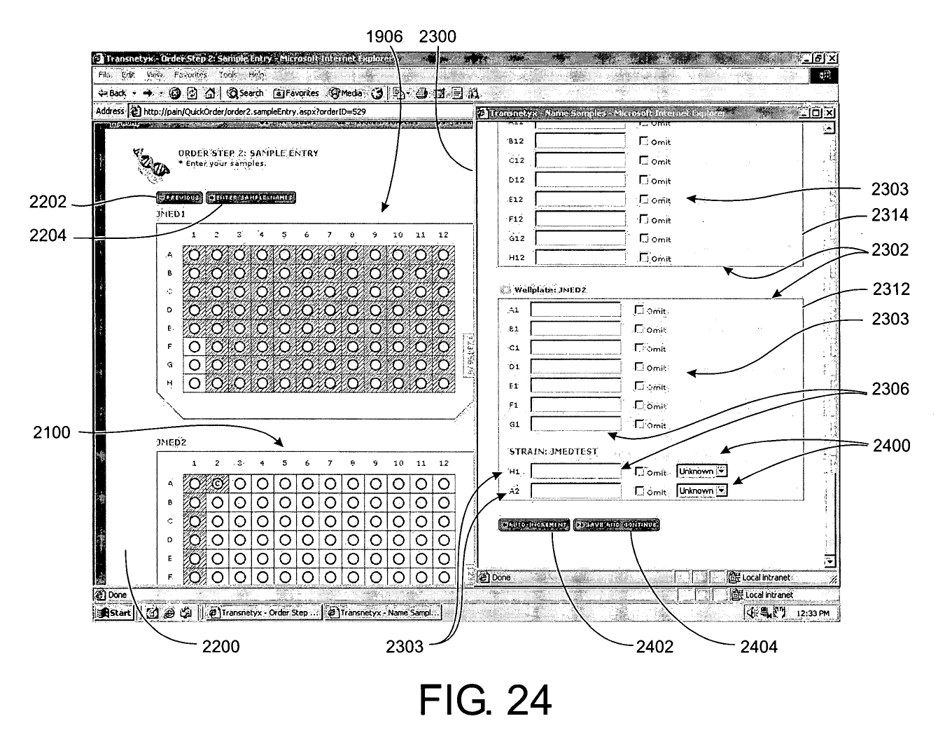

The process for automatically filling these names will be explained in conjunction with the picture of web page 2300 in FIG. 24. In FIG. 24, web page 2300 can be seen as it appears when scrolled to the bottom of the web page. In the bottom portion of sample detail web page 2300 one can see sample list 2302 which includes a second block 2312 of samples set apart from a first block 2314 of samples.

First block of samples 2314 in list 2302 are the samples that are allocated to the first well plate and shown in first well plate image 1906. Web server 102 allocates them to the first well plate image 1906, since they are first on the sample list 1904.

The second block of samples 2312 are the samples that are allocated to the second well plate and are shown in second well plate image 2100. The samples in sample block 2312 are allocated to the second well plate image 2100 only after first well plate image 1906 has received its full allocation of samples. As in the case of the first well plate, web server 106 is configured to begin allocating wells to samples in the second well plate following the same pattern in the first well plate and starting with well location A1.

There are two buttons at the bottom of sample detail web page 2300. The first button 2402, when selected by the user, is configured to transmit a request to web server 106 to calculate and automatically fill (or auto-increment) the names of each sample. The second button 2404, when selected by the user is configured to transmit a command to web server 106 to save the names given to each sample in the order database of web server 106.

To auto increment the names for each of the samples, the user must first enter at least two sample names in to text boxes 2306 before pressing the auto-increment button 2402. Web server 106 is calculated to identify the relationship between the two names, calculate an interval of incrementation between the two names (i.e. the character “distance” between successive sample names), to extrapolate new names for successive text boxes 2306 based upon this interval, and to automatically fill each successive empty text box 2306 with these new names. It fills each successive empty text box in the same order it originally allocated samples to the wells when creating well plate images 1906, 2100. For example, if the first text box 2306 includes the name “AA” and a second, immediately following text box 2306 includes the name “BB”, web server 106 will identify the interval of incrementation between the two names as a single alphabetic character in both the first character position and the second character position, and will extrapolate the name of a third text box immediately following the second text box as “CC”, and successive text boxes as “DD”, “EE”, etc. Similarly, if the first text box includes the name “A1”, and the second text box includes the name “B1”, web server 106 will calculate the incrementation interval of the first character position as one alphabetic character, and the incrementation interval of the second character position as a zero, i.e. no incrementation interval at all. Web server 106 will then extrapolate the name of a third text box immediately following the second text box as “C1” and successive text boxes as “E1”, “F1” etc.

Web server 106 does not erase existing labels. In fact, if, after incrementally filling a succession of empty text boxes with sample names, it meets a text box that is already filled with a sample name, the auto incrementation of names automatically stops. We meeting a text box with a name, web server 106 again checks to see whether auto incrementation can begin again. It finds the next two or more text boxes, identifies the incrementation, and then proceeds to fill successive empty text boxes with sample names.

This process of identifying and auto incrementation interval, filling a succession of following unnamed samples, stopping when reaching a named sample, examining that named sample to see whether it and its following sample identify another incrementation interval, then, again filling any succeeding empty sample name text boxes, is repeated until web server 106 assigns names all the allocated samples, or until it cannot discern an auto incrementation pattern and must stop, leaving succeeding sample name text boxes empty.

The final group of samples in sample detail web page 2300 includes two samples allocated to well positions H1 and A2 that were previously identified as control samples by selecting check box 1930 in web page 1900 when the samples were added to sample list 1904. Web server 106 is configured to identify any samples the user identified as control samples with special indicia superimposed on the well location of that sample in well plate images 1906, 2100. In this case, the letter “C” is the indicia, and it is superimposed on well locations H1 and A2.

Web server 106 is also configured to add pull down list box 2400 in line 2303 indicating the type of control allocated to that well location for every sample identified by the user as a control. Web server 106 is configured to automatically create pull down list box 2400 and populate the list box with a plurality of choices and displayed the list box adjacent to the text box 2306 that is configured to receive the control sample's name.

Controls, as described above, are typically used to validate a probe. A positive control is a control having the genetic sequence sensed by the probe. A negative control is a control that does not have the genetic sequence sensed by the probe. In order to determine the signal levels that would be generated by future positive samples and negative samples (i.e. future samples having and not having, respectively, the genetic sequence of interest that is sensed by the probe or probes associated with the strain of the sample), laboratory 110 should first apply the probes to positive and negative control samples and determined the signal levels (quantitative) or qualitative determinations for each type of sample. Again, the signal level refers to the electronic signal generated by the genotyping test equipment that is used at laboratory to detect the presence or absence of the genetic sequence of interest.

For this reason, the laboratory preferably should be informed whether the control samples are positive or negative samples, whether they have or do not have the genetic sequence of interest. The primary function of list box 2400 is to permit the user to indicate whether the control sample is a positive, negative, heterozygous, homozygous or a wild type sample. For this reason, one of the entries in list box 2400 indicates the sample in the same line 2303 is a positive sample, and another of the entries in list box 2400 indicates that the sample is a negative sample. The remote user indicates the type of each control sample by selecting the appropriate entry from list box 2400.

If the remote user does not know whether the control sample is positive or negative, she indicates her ignorance of this fact by leaving list box 2400 with its default selection generated by web server 106, the “Unknown” selection illustrated in FIG. 24.

Some tests performed on biological samples indicate the “zygosity” of the sample. A discussion of zygosity is beyond the scope of this application. However, a biological sample may have three different states of zygosity, commonly called heterozygous, homozygous and wild type samples, which are commonly identified as “++” (plus-plus), “+−” (plus-minus), and “−−” (minus-minus). For this reason, list box 2400 also includes three additional entries corresponding to these three different states of zygosity.

Once the user has finished identifying each of the samples previously allocated to well plate images 1906, 2100 and shown as groups in sample list 1904 and as individual samples in list 2302, the user can complete her order. The user does this by clicking button 2404, which commands web server 106 to save the sample names and the type of control sample they are (if the sample is a control sample) in its order database. Web server 106 responds by generating a new sample entry web page 2200 in which each well in well plate images 1906, 2100 is overlaid with an indicia indicating that the well has been named. These indicia, as best shown in legend 2206 in FIG. 22 include a darkened central region to the well. See, for example, legend entry 2406 in FIG. 22. Only the samples that have actually been given sample names are changed to have a darkened central region when the user clicks button 2404 (FIG. 24) and saves the newly added or revised sample names to web server 106.

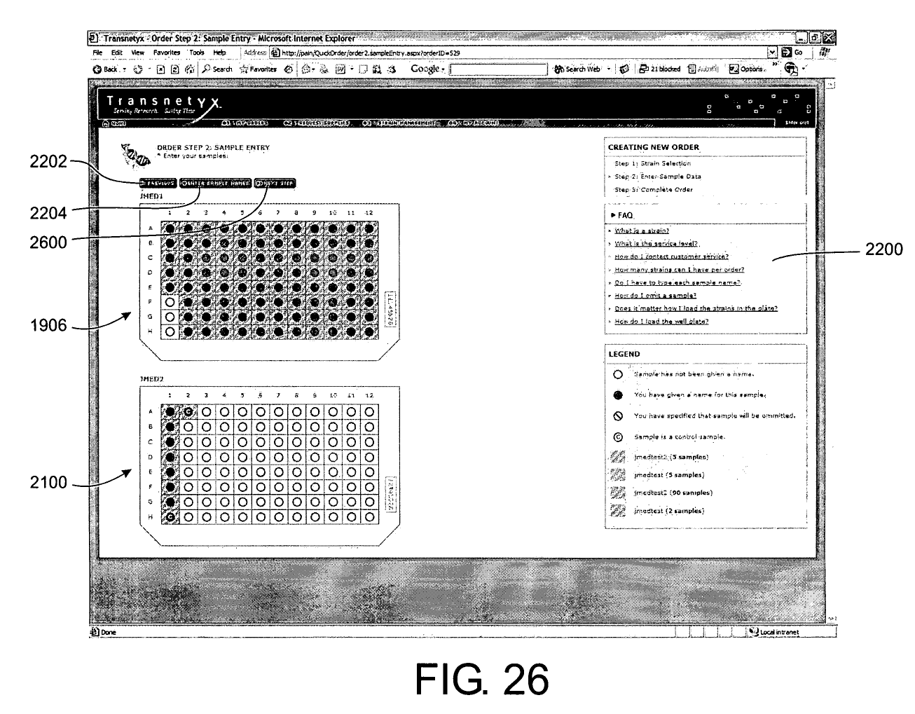

Once the user has saved all the sample names to web server 106 by clicking button 2404, web server 106 generates a new sample entry web page 2200 showing all of the named wells in well plate images 1906 and 2100 with darkened central portions to indicate that all the wells have been named. By indicating which wells have been named in well plate images 1906, 2100 (which are graphical representations of two containers 502 that the remote user will fill with samples), the remote user can visually compare the names with the samples actually placed in the wells directly. This arrangement is best shown in FIG. 26.

Referring now to FIG. 26, note that all the wells in the two well plates 1906, 2100 that contain samples are darkened, and that well locations identified as not containing samples (“empty samples” wells F1, G1, H1) are not shown darkened. This serves as a reminder to the remote user that intentionally empty wells do not have samples. It further provides an easy way for the user to examine the well plates filled with samples and assure herself that no samples have been placed in wells that are not intended to contain samples. By providing well images that indicate not only the empty wells, but the filled wells, the remote user can make these visual comparisons quite easily by a simple and fast visual comparison of the well plate images 1906, 2100 with the actual well plates (i.e. containers 502) in which the user will place or has placed the biological samples for testing.

Web server 106 has generated an additional remote user selectable item, namely button 2600, which is located adjacent to buttons 2202 and 2204, in its revised sample entry web page 2200. Web server 106 does not automatically create this button whenever the remote user fills in one or more names for each sample shown in the well plate images. Instead, web server 106 only displays this button (and only gives the user the choice represented by the button) when the user has created and entered a name for every sample in well plate images 1906, 2100. Whenever the user clicks button 2404 in sample detail web page 2300, the user transmits the data she entered in that page to web server 106. Web server 106, responsively checks each of the sample names submitted by the user in text boxes 2306 to make sure that no box has been left empty (i.e. no sample name has been provided for a sample).

If, after this comparison, web server 106 determines that all samples allocated to the two well plate images have been provided with associated names by the user, web server 106 responsively generates button 2600. Button 2600 permits the user to continue on to the next step of the ordering process, in which the user places an actual order with web server 106 for biological testing (e.g. genotyping) the samples. On the other hand, if web server 106 determines that all samples shown in the well plate images are not provided with a name, it will merely close window 2300, update all the well locations in the well plate images for which names have been provided by illustrating them with the darkened central portion in well plate images 1906, 2100, and wait for the user to click on button 2204 to enter additional names. In this case button 2600 will not appear on the screen and the user will not have the choice of continuing onward to place an order. In an alternative embodiment, the widget or other selectable item could be placed on web page 2300 or sample entry web page 2200 and not be specially generated by web server 106. In this alternative case, however, web server 106 would still perform its verification that all sample names have been provided, and would refuse to continue the ordering process and actually place an order if all the names were not provided. The button is provided when the order is complete to provide the user with a graphical indication that she is not finished adding sample names and for that reason cannot go further. Of course, web server 106 can be configured to generate an audible or graphical notification to the user that not all samples have been named, for example by having remote user computer 102, 104 beep, or open a small message box or alert box with a graphical (textual) indication that sample naming is not complete.

If user wishes to change the order, even after all of the well locations have been named (as shown, for example, in FIG. 26), the user can simply reopen web page 2300 by clicking on button 2204 in sample entry web page 2200. Even after the order has been complete, web server 106 will open web page 2300 with all of the data filled in exactly as the user entered it originally. The user can simply edit this data and then click again on button 2404 in web page 2300 to close web page 2300, its window, automatically saving the edited data to web browser 106, which will then automatically regenerate sample entry web page 2200 to reflect this amended or added data.

When the user selects button 2600, web server 106 will replace web page 2200 with order finalizing web page 2700, best shown in FIG. 27. Web page 2700 illustrates the final step in the ordering process, that of finalizing the order and placing it with (submitting it to) web server 106. Web page 2700 includes text region 2702 which shows the shipping address to which the samples in container's 502 are to be sent. This is preferably the address of the laboratory 110 that actually performs the testing.

Web page 2700 also includes text region 2704 which shows the billing address to which the bill for the sample testing shall be sent. Web server 106 preferably generates this text from data previously typed into account management web page 2900.

Web page 2700 also includes a text box 2706 in which the user enters a character string that identifies the package in which the samples are sent.

Typically, the character string includes an order number, tracking number, billing codes, or barcode number that can be used to access an electronic shipment database maintained by the shipping company to track the shipment of samples. In the preferred embodiment, the number is a FedEx order number or tracking number that can be entered at the FedEx web site (www.fedex.com) to identify the location of the package containing the samples at all times during its shipment between the user and laboratory 110. Of course, similar order or tracking numbers for other freight services having a web package tracking application are also preferred.

In the preferred embodiment, each box 501 in which empty sample container 502 was delivered to the user is also used as they return package for shipping the now-filled container (or containers, in the example above) 502 back to the laboratory 110. The character string typed in the text box 2706 is preferably also printed on a shipping label that the user fixes to the outside of box 501 for shipment to laboratory 110.

Web page 2700 also includes a text box configured to receive an expected ship date typed in by the user. This expected ship date is transmitted to personnel at laboratory 110 to permit them to make last-minute adjustments in their schedule, and to ensure that they have supplies and staff available to service the order in the agreed upon time.

Web page 2700 includes a text region 2710 that indicates the agreed upon time for servicing the order and for returning the results to the end-user. This data was previously entered in text box 1902 in web page 1900 (FIG. 19). In this example, the service-level is 72 hours.

Web page 2700 also includes an order confirmation list 2712 which lists the individual components of the order, the price of each component, and the total price of the order. The components of the order include several lines, each line representing one or more samples sharing common testing characteristics. In the preferred embodiment, each group of samples entered into the sample list 1904 is listed in its own line, since each line in the sample list 1904 can have a different strain, with a different number of probes, and a different number of samples tested for that strain with those probes, each line in the order confirmation list is broken down in the same manner, listing the strain 2714 for which the biological testing is to be done, the number of probes 2716 that are used for that test, the number of samples 2718 that are to be tested, and the service-level 2720 for those samples. These are the common testing characteristics of each group of samples in sample list 1904. Each line in the order confirmation list 2712 also includes an associated price 2722 for testing the group of samples identified in that line.

An advantage of this format is that it permits multiple tests for multiple strains and for multiple researchers and research projects to be transmitted to research lab 110 in a single multi-well plate container 502, yet also preserves the individual billing data on a sub-plate level (i.e. individual billing information for groups of samples numbering less than the total number of samples possible in the multiple well plate container 502). By providing the user with individual order information for each group of samples, samples from several different groups can be conglomerated and sent in a single container 502. This reduces both shipping costs and processing time, since each researcher does not have to prepare and transmit his own order in order to have his own bill. The bill is broken out by individual strains, and groups of strains. Any time handling is reduced and data entry is reduced, errors are reduced.

Order confirmation list 2712 also includes an individual fee for handling the well plate itself 2724. This fee represents the costs of transmitting package 500 material to the user, the cost of handling box 501 when it is returned to laboratory 110, and handling the container 502 when it is removed from the box and prepared for testing. In the preferred embodiment, cost 2722 for each individual group of samples represents a multiple of a base cost for each sample in that group. For example, two probes are used for five samples in the first group shown in line 2726 in list 2712. The cost, $75, represents the individual sample cost of $15 for the two probes used in the test; times the five samples that are tested. Similarly, for example, the cost of $125 for the five samples tested with four probes in second line 2728 of list 2712 represent the individual sample cost of $10 for two of the probes used in the test plus an additional cost of $15 for the other two probes used in the test, times the five samples that are tested. $10 plus $15 times five equals $125. Order confirmation list 2712 therefore permits the user to determine the cost of each individual sample that is tested, based upon the number of probes that are tested, and to allocate that cost to individual researchers or research projects, when several researchers or research projects are combined in a single order. Even if the researchers are performing identical tests (i.e. testing for identical strains using identical probes), the remote user can make sure their costs are separately calculated by adding each researchers samples, as a separate item in the sample list 1904. This can be seen in the present bill, in which there are samples tested for two strains, but each strain is divided into two separate cost entries. Thus there are two cost entries for strain “jmedtest” and two cost entries for strain jmedtest2”. This is particularly advantageous when used with large institutions such as universities or pharmaceutical companies. A single clerk can be assigned a job or preparing an order for biological testing once a week, for example, and can accept individual suborders from multiple researchers within his institution. One single container 502 can incorporate several of these individual suborders, each suborder being shown on a separate line of the order confirmation list 2712. In an extreme case, there could be as many cost entries in the bill as there are wells in the well plate. Since an entire well plate can be filled with samples that are allocated as many different ways is necessary to create individual line items in the bill, the remote user need not take each researcher's samples and place each researcher's samples in a separate well plate dedicated to that researcher. While this would certainly generate separate bills, increases shipping costs, increase is processing time at laboratory 110, and increases the probability for error. Thus, only one data entry session at remote computer 102,104 is required for the remote user to prepare several individual orders, only one container 502 is required to transmit several individual orders, and only one shipping cost will be incurred to ship all the individual suborders.

Order confirmation list 2712 also includes a subtotal 2726 which shows all the individual sample group costs and the well plate costs 2724. Order confirmation list 2712 also includes a shipping cost 2728 and the total cost 2730 of all the above listed costs.

Once the user has mentally affirmed these costs she can press the button 2732, which confirms the order and formally submits the now-completed order to web server 106. At this point, the order has been placed. Once it receives a submitted order, web server 106 is configured to send notice to personnel at laboratory 110 that a new order has been placed and that lab 110 will be receiving a shipment sometime shortly after the user's expected ship date 2708.

Once remote user has pressed button 2732, her messages are transmitted to web server 106, and web server 106 responsively transmits an order verification web page 2800 back to the user. Order verification web page 2800 provides visual confirmation 2802 to the user that the order has been submitted successfully to web server 106, which then submits it to LIMS 108 and laboratory 110. Confirmation 2802 is preferably a few statements indicating successful submission of the order, and in this case preferably includes an identification 2804 of the two containers 502 that web server 106 understands the samples will be shipped in. Identification 2804 preferably includes indicia 503 affixed to container 502, which uniquely identifies one container 502 to web server 106 and LIMS 108.

In some cases, the user may not wish to actually submit the order to web server 106 even after all the order information has been added. The remote user may not be authorized to place orders, for example, and thus may wait for a third-party to examine the order, confirm that it is correct, and then press button 2600 to place the order and button 2732 to confirm the order.

To permit the user or other personnel to return to the order and complete at a different time, web server 106 maintains an order database that identifies the order, the samples to be tested the individual tests to be performed on the samples, the containers 502 and indicia 503 of the containers in which the samples will be sent, the strains for which the testing is to be done, the probes for which each strain will be tested, and the location of each individual sample in the containers.

The order database includes this data for all orders, including orders that are incomplete (e.g. orders that have not yet been placed with web server 106), those that are being processed (e.g. orders that have been placed with web server 106), and those that have been fulfilled by laboratory 110 (e.g. orders which laboratory 110 has finished testing and has reported out test results to web server 106).

To access this order database, view any of the orders listed therein, and submit any incomplete orders to web server 106, the user returns to main web page 900 and clicks on button 902. When the user does this, web server 106 responsively generates web page 2500, best shown in FIG. 25.

Referring now to FIG. 25, once the user selects button 902, web server 106 responsively generates order home page 2500. Order page 2500 several widgets, including a list 2502 of all orders, including incomplete, complete and fulfilled orders. Each line 2504 in order list 2502 includes a check box 2506 for selecting the order on that line, a label 2508 indicating when the order was last modified, a label 2510 indicating the total number of strains and the total number of different probes identified in that order, a label 2512 identifying the total number of samples in the order, and a label 2514 indicating the status of the order as incomplete or complete. At the bottom of list box 2502 is a button 2516 which, when clicked on by the user, deletes any order in list 2502 whose check box 2506 the user has checked and is currently checked. To reduce the chance for errors, when web server 106 generates web page 2500 it always generates the page with check boxes 2506 unchecked.

Each line in the order list 2502 represents an order that has been at least partially prepared. An order is deemed to be prepared enough to be incomplete and listed in the order list 2502 when at least one strain has been added to an order by clicking button 1924 on web page 1900 (FIG. 19). Button 1924 transmits the first item of information for a new order to web server 106. Each successive submission of data to web server 106 signals web server 106 to add the additional data to the incomplete order. Orders are maintained in web server 106 until they are explicitly removed by selecting checkbox 2506 adjacent to that order and button 2516 which deletes that order.

In addition to remembering the data previously submitted by the user to web server 106, web server 106 also stores data identifying the last web page viewed by the user. This data permit the user to return to the most recent stage of the ordering process, the stage she was last at when she exited the incomplete order.

When the user views the order list 2502, she can select an order and immediately jump to the last web page in the order process by selecting or clicking a button associated with that order. In the preferred embodiment, the button that she can select is label 2508, which indicates when the (incomplete) order was last modified. When the user clicks on label 2508, she signals web server 106 to locate that order in the order database in web server 106, identify the last order page generated by web server 106, and to regenerate that web page and transmit it to the user at remote user computer 102, 104 for display by the user's Web browser. This saves the user the added time necessary to navigate web page 1900 and subsequent order web pages, if the last page viewed was web page 2700 (for example). Instead, she can immediately jump to web page 2700, the page at which the user submits the order to web server 106. Of course, merely presenting the user with web page 2700 immediately, does not automatically place the incomplete order. She can still navigate the web site before placing the order. She can navigate through the incomplete order itself to change any erroneous data or add new data by clicking on the “previous” buttons in each order web page, such as button 2734 in web page 2700. These buttons, identified in the present application as “previous”, permit the user to back through any data entry process by signaling web server 106 to regenerate the previous web page.

Referring back to FIG. 27, the order data also included a shipping address and a billing address. Web server 106 requires that this account information be provided by the user before it will permit the user to submit an order by clicking on button 2732. The user enters this account information in the account management web pages which are generated by web server 106 whenever the user selects button 908 on main web page 900.

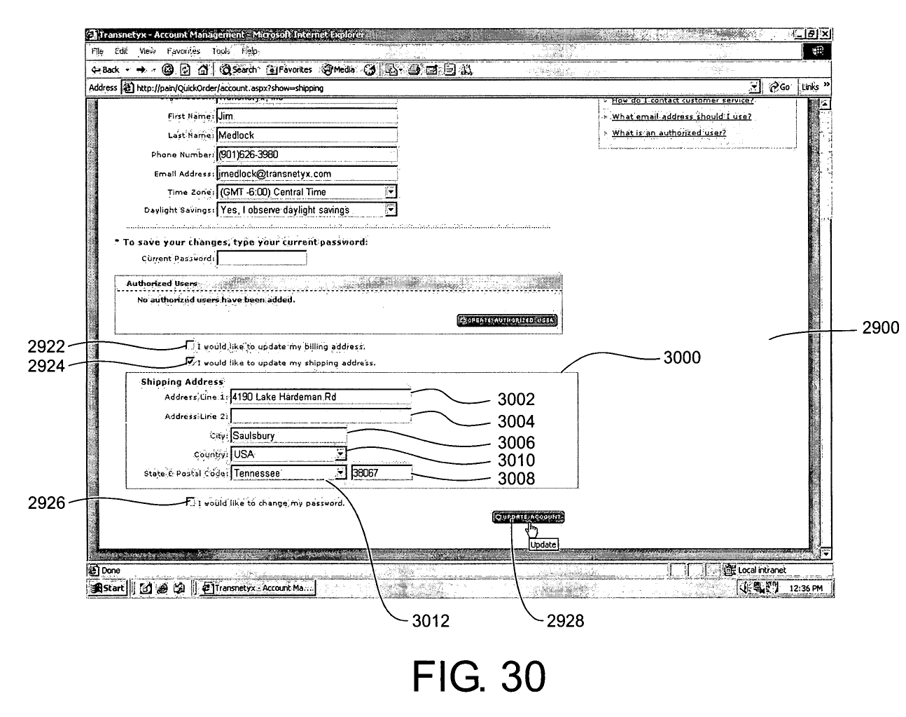

FIGS. 29 and 30 illustrate the account management web page 2900. Web page 2900 is generated by web server 106 to provide a convenient interface to basic account data. Web page 2900 includes basic account information identifying the remote user. This includes text box 2902 for entering and displaying the organization name, text boxes 2904 and 2906 for entering the first and last name of the remote user, text box 2908 for entering the remote users are organizations telephone number, text box 2910 for entering the remote users e-mail address, pulldown list box 2912 includes several time zones from which the user may select the time zone from which the remote user will operate the remote user computer 102, 104, and pulldown list box 2914 from which the user may select whether or not he observes daylight savings time.

Account management web page 2900 also includes a text box 2916 in which the user can enter his current password to prevent unauthorized people from making changes to the basic account information, above, when the user leaves remote computer 102,104 temporarily unattended.

Account management web page 2900 also includes a list box 2918 list any authorized users that the remote user has created. Authorized users (or sub-users as they are also known) are users that are created by the remote user who have the authority to create strains and create orders. Each authorized user created by the remote user is added to a user database maintained by web server 106. Each authorized user is given an account name and password different from the account name and password of the remote user. The authorized users can log on to web server 106 in exactly the same manner that the remote user does, can create strains and orders, and can save the strains and orders in precisely the same manner as the remote user. What each authorized user cannot do is view any order or strain created by either the remote user, or by any other authorized user created by the remote user. The authorized user also cannot change any of the account information entered by the remote user and displayed in account management web page 2900. This arrangement permits each authorized user to access web server 106, create strains and create orders which will be charged to the account created by the remote user. The remote user can always see the strains created by every authorized user by going to the strain management page (FIGS. 10 and 14) and viewing the strain list 1402. Strain list 1402 lists not only the strains created by the remote user, but the strains created by all of the authorized users as well. Similarly, the remote user can always see the orders created by every authorized user by going to the order management web page 2500, and viewing the order list 2502. To prevent each authorized user from viewing and modifying other authorized user's (and the remote user's) data, web server 106 is configured to identify whether a user logging on to web server 106 is a remote user (i.e. a master user having full access to the account) or whether the user is an authorized user (with limited access to the account). Web server 106 tailors the data that it displays to each user when web server 106 generates the various web pages identified herein preventing each authorized user from viewing strains, probes, and orders created by any other user, and permitting each authorized user to view their own strains, probes, and orders. Web server 106 likewise tailors the data to permit the remote user (i.e. the master user) to view every strain, probe, and order associated with the account, both those created by all authorized users and by the remote user, and also to permit the remote user to change strains and orders that are created using the remote user password and account name.

The remote user can view all of the strains and orders created by all of the authorized users. The remote user can change the authorized users' orders, or submit the authorized users' orders, or change the authorized users' strains. The primary role of the remote user is to administer the account, and correct errors and change data entered by individuals logged in as different authorized users. For this reason, the remote user must have access to view and modifying all of the sub-users and their activity, which it is given by web server 106.

To add an authorized user (i.e. sub-user) the remote user clicks on button 2920 which is displayed in the bottom of user list 2918. When the remote user clicks on button 2920, the remote user computer 102,104 is configured to transmit a signal to web server 106, indicating that the remote user would like to create an authorized user. In response, web server 106 generates a pop-up window displaying the create user web page 3200 (see FIG. 32).

Create user web page 3200 includes a text box 3202 for entering a new authorized users first name, a text box 3204 for entering a new authorized users last name, and a text box 3206 for entering the e-mail address of the new authorized user. Web page 3200 also includes a submission button 3208, which, when pressed signals remote user computer 102,104 to transmit the data in web page 3202 web server 106. Web server 106 responsively creates an e-mail message that it sends to the e-mail address indicated in text box 3206.

This e-mail message offers the new authorized user the opportunity to create an authorized user identity on web server 106, which will allow the authorized user to create strains, probes and orders in the remote user's account, and will also permit the authorized user to submit those orders to web server 106 in the same manner described above for the remote user. It indicates the domain name of web server 106, indicates the account name the proposed new authorized user will use (preferably her e-mail address), and indicates the password associated with that account name. Web server 106 is configured to generate passwords whenever a remote user creates a new authorized user. This password is not communicated to the remote user, and therefore the remote user cannot access web server 106 by assuming the identity of another authorized user.

When web server 106 receives the name and e-mail address of the proposed new authorized user, it also creates an entry in the user database in web server 106. This database associates the name and e-mail address of the new authorized user proposed by the remote user with the remote user's account. It also indicates that the proposed remote user is not yet registered—i.e. has not yet logged on to web server 106 a first time to gain access.

Once the remote user has requested the creation of a new authorized user, whenever web server 106 transmits account management web page 2900, it will update it, showing the new remote user in the authorized user list 2918. An example of a new authorized user that has been added to authorized user list 2918 (but has not yet become registered by logging on to web server 106 a first time) is shown in FIG. 32. In that figure, a typical authorized user is shown in the authorized user list 2918. He is listed on a single line 3210 in list 2918 that includes his first and last name 3212 on the left-hand end of line 3210, and his status 3214 in the middle of line 3210. The new authorized user's first name is “Jim”, and his last name is “Medlock”. His status is “Not Registered” which indicates to the remote user viewing this page that the new authorized user has not yet responded to the e-mail message and logged on to web server 106.

Logging on to the system is essential to becoming a full participant in the remote user's account. Once a proposed new authorized user logs on to web server 106 using the account name and password provided by web server 106 in the e-mail message, web server 106 responds by changing the status of that authorized user to “Registered”. Thenceforth, when the remote user reconnects to web server 106, logs on, and displays web page 2900 again, web server 106 will display that authorized user with their new status of “Registered”.

Below the authorized user list 2918 in FIG. 29 are three check boxes 2922, 2924 and 2926. The remote user can check any or all of these boxes to indicate his desire to update the remote user billing address, update the remote user shipping address, or change the remote user password.

When the remote user has updated the account information in each of the text boxes and list boxes 2902-2914, or has alternatively check any of check boxes 2922-2926, the remote user then transmits this updated information to web server 106 by clicking on button 2928. Button 2928, when clicked by the user, transmits the new information to web server 106, which updates all of the modified data. Web server 106 also regenerates web page 2900 and transmits it to the remote user for display on remote user computer 102,104.

When web server 106 regenerates web page 2900 in response to the user clicking button 2928, the web server creates an address block 3000 that is disposed below check box 2924 if check box 2924 was checked. The web server also creates another address block (not shown) that is disposed below check box 2922, if check box 2922 was checked. The two address blocks include address information for the billing address (located below check box 2922) and the shipping address (located below check box 2924). This address information is presented in text boxes 3002, 3004, 3006, 3008, pulldown list box 3010 and pulldown list box 3012. Text boxes 3002 and 3004 are for address lines one and two of the address. Text box 3006 is for the city. Text box 3008 is for the postal code. Pulldown list box 3010 is for the country, and pulldown list box 3012 is for the state. Pulldown list box 3010 is populated by web server 106 with a plurality of the countries of the world among which the user can select the billing address country. Pulldown list box 3012 is populated by web server 106 with all the states of the United States. The description in the previous paragraph explains the contents of the shipping address block. The billing address block (not shown) that is presented by web server 106 below check box 2922 when billing address check box 2922 is checked includes the same items formatted in the same manner as shown in FIG. 30.

Whenever the user fills a sample container 502 and ships it to laboratory 110, this container must be replaced in the user's inventory. To do this, the remote user returns to main web page 900 and clicks on button 904. Button 904 signals web server 106 to generate supply request web page 3100, best shown in FIG. 31. Alternatively, the user can go directly to web page 3100 by clicking on the “request supplies” button

Referring now to FIG. 31, the supply request web page 3100 includes the shipping address of the remote user 3102 which is by the remote user to ensure the remote user is aware of the shipping address. If the shipping address is incorrect, the remote user can correct the shipping address by selecting link 3104, which takes the user to the account management web page 2900 to make any necessary changes.

Supply request web page 3100 also includes a text box 3106 in which the remote user enters the number of packages 500 (or “lab kits” as they are identified in web page 3100). Supply request web page 3100 also includes a drop-down list box 3108 that is populated by web server 106 to include several alternative shipping choices that differ by the speed of delivery. One choice in the list is preferably overnight shipment for arrival the next morning. One choice in the list is preferably overnight shipment with delivery the next afternoon. One choice is preferably 2 day delivery. And one choice is preferably 4-5 day delivery.

Finally, supply request web page 3100 includes a button 3110 that the user clicks to transmit his request for supplies to web server 106. When web server 106 receives the request for supplies, including the number of lab kits and the method by which they should be shipped, it generates a work order for operators at laboratory 110 to prepare a shipment of packages 500 and transmit them to the remote user at her shipping address for delivery in the time selected by the user in box 3108.

Eventually, laboratory 110 will perform the tests indicated in the order that the remote user placed. When the tests on each of the samples are complete, the results are gathered by LIMS 108 and compiled. LIMS 108 then sends the test results to web server 106, which enters them in its order database.

When web server 106 receives the test results, it is configured to automatically generate an e-mail message and transmit that e-mail message via e-mail to the user that place the order, in this case the remote user. The message (not shown herein) typically tells the remote user to go to the URL identifying web server 106, and to log on. The remote user, receiving the e-mail, responds by connecting to web server 106 (root web page 700) and logging on, at which time web server 106 presents the remote user with main web page 900.

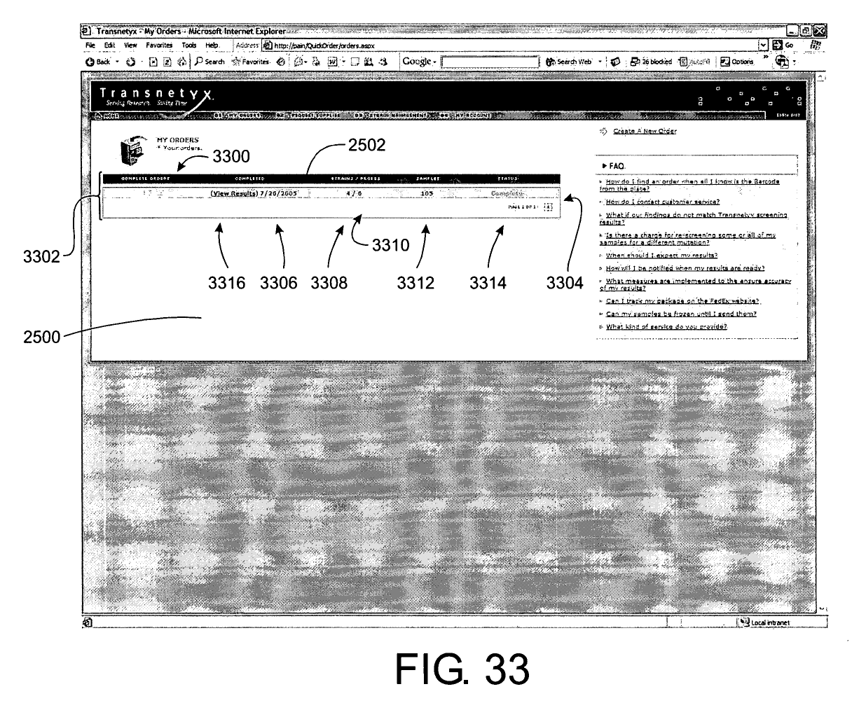

The remote user then selects any of the “My Orders” buttons 902 or 914, where upon web server 108 will generate a revised orders web page 2500 (FIG. 33). Using the example from above, the orders web page originally had an order identified as “incomplete” because the order had not yet been submitted or placed with web server 106. In the example above, we describe how the remote user places the order. When the test results are returned, web server 106 changes the status of that order in its order database to “complete” which signals web server 106 to display the order differently on orders web page 2500, when the remote user next generates that web page. In particular, all completed orders, such as the order created, placed, and completed by the remote user, as described above, appear in order list 2502 as a “complete” order, and not an “incomplete” order. Furthermore, completed orders appear in an order list 2502 having a header identified with the label 3300 “complete orders”. Note that if there were incomplete orders (orders that have not yet been placed), or pending orders (orders that had been placed, but for whom the testing is not complete), web server 106 would generate these orders in a separate list block 3302 with the labels “pending” and “incomplete” to indicate the status of these orders.

The completed order appears as a single line 3304 in order list 2502. This line lists the date 3306 the order was completed, the number of strains 3308 in the order, the number of probes 3310 in the order, the number of samples 3312 in the order, and the completion status 3314 of the order as “complete”, “pending”, or “incomplete”.

Line 3304 provides a selectable link 3316 that the remote user can click to view the results of the test. This link is shown in line 3304 as hypertext link “View Results”. When the remote user clicks on link 3316, remote user computer 102,104 transmits a request to web server 106 to display the test results associated with that order. Web server 106 responsively generates a pop-up window showing test results web page 3400 listing test results 3402.

Test results 3402 include contact information 3404 for the entity that provides the testing, including that entity's name, address, phone number, and fax number. Below that, test results 3402 display contact information 3406 for the remote user, including the remote user's name, the entity for which the testing is performed (typically the remote user's employer), and its address.

Test results 3402 also include several dates 3408, including the date and time the order was submitted to web server 106, the date and time the container or containers 502 holding the samples described in the order were received at laboratory 110, and the date and time the testing of those samples was completed.

Test results 3402 also include a selectable link 3410 which the operator can click to request instructions explaining the test results. Web server 106 is configured to open another pop-up window (not shown) in response to this request and display an explanation of the results. The explanation of the results will include a description of what each column in the test result list represents, and what special symbols represent, for example.

Test results 3402 also include a result list 3412 that is divided into several blocks of results 3414, 3416 and 3418. Each of these blocks includes all the samples identified in one of the sample groups in sample list 1904 of web page 1900. Each sample group in the sample list corresponds to a single line in that list, identifying the strain and the number of samples associated with that strain. Since the remote user identified the samples in these groupings, providing the test results in the same groupings will assist the remote user in associating the results with the samples.

Each result block 3414, 3416 and 3418 includes a header line 3420, 3422, 3424, respectively, that lists the name of the strain, and the names of each of the probes that were selected for use with that strain. In the first result block 3414, a container 502 identifier, preferably identifier 503, is also located to indicate the beginning of the samples in that container.

Header line 3420 of the first block 3414 includes name 3426 of the strain for which that group of samples was tested, and the names 3428, 3430 of the two individual probes, Kodi KO, and Lac Z, respectively, that the remote user selected for testing. Web server 106 is configured to spaces the probe names horizontally apart such that the individual test results can be disposed in a column beneath the probe names. The number of probes for each block of samples will vary, depending upon how many probes the remote user identified for use with that strain. Web server 106 is configured to automatically adjust the probe name-to-probe name spacing between each of the probes in the header line to space them equally apart for improved readability.

Each sample is listed on a separate line in result list 3412. Each line includes indicia 2304 identifying the unique location of the sample in container 502 (also in the well plate image 1906 corresponding to container 502), the name 3432 of the sample that either the user typed into text box 2306 or web server 106 automatically filled in text box 2306 with its auto-fill programming.

When the remote user initially created the order, the remote user transmitted the order information to web server 106, which saved the order information in its order database. This data includes the indicia 503 of container 502 in which the sample was inserted, and the location 2304 of the sample within container 502. Web server 106 is configured to automatically retrieve this information from its order database and to incorporate this information into the test results 3402.

Each line in results list 3412 also includes indicia 3434 and 3436 indicating the results of the testing for each of the probes. In the case illustrated herein of first block 3414, the probes only have two possible states, positive, and negative, that are be reported. The testing determines whether the sample is positive or negative for these probes. Hence, the indicia used to indicate the results is a “+” for positive result and “−” for negative result. In this sense, a positive result means that the sample had the designated genetic sequence that the probe is configured to bond with. A negative result means that the sample did not have the designated genetic sequence that the probe is configured to bond with. The indicia 3434 are preferably organized in a vertical column underneath the name 3428 of their associated probe. Similarly, the indicia 3436 are preferably organized in a vertical column underneath the name 3430 of their associated probe. This organization makes it easy for the user to usually scan through many samples quickly.

In the order identified herein, two containers 502, shown graphically in the web pages above as well plate images 1906, 2100, were used to transport the samples to laboratory 110. Test results 3402 indicate the point at which one well plate quits and the next plate begins by inserting and indicia 503 for the second well plate into the test results immediately before the second well plate samples are listed. The test results 3402 illustrated in window 3400 list all 103 well locations and the test results of the 100 samples in those locations in list 3412 in the manner described above with regard to the first group 3414 of samples. To view these additional samples, however, the user must scroll window 3400 downward until those samples are scrolled into the user's view.

From the foregoing, it will be observed that numerous modifications and variations can be effected without departing from the true spirit and scope of the novel concept of the present invention. It will be appreciated that the present disclosure is intended as an exemplification of the invention, and is not intended to limit the invention to the specific embodiment illustrated. The disclosure is intended to cover by the appended claims all such modifications as fall within the scope of the claims.

For example, although particular databases have been identified for web server 106 and LIMS 108, the data in these databases can be combined into fewer databases, including a single database, or can be further subdivided into a greater number of sub-databases. Furthermore, individual databases can be combined, and elements of each database can be moved from one database to another. Thus, a portion of any of the described databases can be incorporated into another database.

As another example, LIMS 108 is shown located at laboratory 110. LIMS 108 need not be located at laboratory 110, however, but can be located elsewhere and in communication with laboratory 110 and with web server 106 over a LAN, WAN, or the Internet.

As yet another example, the computers and servers 102,104,106,108 described herein are each shown as software and data existing on a single computers. In an alternative embodiment, each may be comprised of multiple computers, with each computer performing a portion of the functions identified for the computers and servers.

Even further, while the description above relates to a particularly preferred application of biological testing, that of genotyping using strains and probes, the identical process can be used with other forms of biological testing, and even genotyping using profiles and primer sets. In such case, instead of having a strain database and manipulating strains, and a probe database and manipulating probes, web server 106 would have a profile database and manipulate profiles (in place of the strains, above), and would have a primer set database and manipulate primer sets (in place of the probes, above).

In the preferred embodiment illustrated above, the user at remote user computer 102,104 interacts with a web server 106. Web communication over the Internet is preferred mode of placing an order. Dedicated programs, however, can communicate the same information between remote user computer 102,104 and another computer such as web server 106, that need not be configured to communicate with remote user computer 102,104 as a web server, however. Instead, dedicated programs operating on computer 106 can communicate the same information back and forth between remote user computer 102,104 and computer 106 over the Internet.

If LIMS 108 is not located at test laboratory 110, additional computers at test laboratory 110 can be configured to communicate over network 112 with LIMS 108, to communicate information to LIMS 108 such as the identifiers or indicia on boxes and containers shipped to testing lab 110 from the remote user and containing her order.

In the preferred embodiment illustrated above, all the web pages are described as being generated by “web server 106”. In an alternative embodiment, web server 106 may be a plurality of individual web servers each providing a different web page. In another alternative embodiment, these individual web servers may be disposed at different locations, and communication between the remote user computer 102,104 and web server 106 may include transmissions from remote user computer 102,104 to two or more different physical computers located at two or more different locations, each one of which can perform any one or more of the functions described above as being performed by web server 106.

1. A computer-implemented method for ordering biological tests for biological samples, the method comprising:
electronically selecting a first strain for testing;
electronically selecting a first plurality of biological samples in a first sample order to be tested for the first strain;
electronically transmitting over the Internet the first strain associated with the first plurality of biological samples to a computer configured to receive orders for biological tests; and
sending the first plurality of samples to a testing laboratory in a first package.

2. The method of claim 1, wherein:
the step of electronically selecting a first strain for testing includes the step of electronically selecting a second strain for testing;
the step of electronically selecting a first plurality of biological samples includes the step of electronically selecting a second plurality of biological samples to be tested for the second strain;
the step of electronically transmitting includes the step of electronically transmitting over the Internet the second strain associated with the second plurality to the computer; and
the step of sending the first plurality includes the step of sending the second plurality of samples to the testing laboratory in the first package.

3. The method of claim 1, further comprising:
labeling the first package with a preaddressed label having first indicia before the step of sending;
wherein the step of electronically transmitting the first strain includes the step of electronically transmitting the first indicia to the computer over the Internet.

4. The method of claim 3, wherein the preaddressed label includes at least one identifier directing the shipping company handling the first package to automatically charge shipping costs to a first entity other than the entity performing the steps of claim 3.

5. The method of claim 4, wherein the first entity is the entity that tests the first plurality of samples.

6. The method of claim 1, further comprising the steps of:
placing the first plurality of samples in a single multiwell container; and
placing the single multiwell container into the first package before the step of sending the first plurality of samples.

7. The method of claim 2, further comprising the steps of:
placing the first and second pluralities of samples into a single multiwell container; and
placing the single multiwell container into the first package before the steps of sending the first and second pluralities of samples.

8. The method of claim 1, further comprising the steps of:
electronically creating the first strain by electronically storing a name of the first strain; and
electronically storing a plurality of probes in association with the name of the first strain.

9. The method of claim 8, further comprising the steps of:
electronically transmitting the created first strain to the computer.

10. The method of claim 1, further comprising the steps of:
electronically selecting a first probe from a plurality of probes; and
electronically associating the first probe with the first strain.

11. The method of claim 10, further comprising the steps of:
electronically selecting a second probe from the plurality of probes; and
electronically associating the second probe with the first strain.

12. The method of claim 1, wherein the first strain includes a plurality of probes, the method further comprising the step of electronically deselecting at least one of the plurality of probes from the first strain.

13. The method of claim 1, further comprising the steps of:
electronically associating the first plurality of biological samples with the first strain for testing; and
signaling the computer to automatically allocate the first plurality of biological samples to a corresponding first plurality of wells in a multiwell container in accordance with an electronically predetermined pattern of well filling;
wherein the step of sending the first plurality of samples to the testing laboratory in the first package includes the step of placing the multiwell container in the package.

14. The method of claim 2, further comprising the steps of:
electronically associating the first plurality of biological samples with the first strain for testing;
electronically associating the second plurality of biological samples with the second strain for testing after the step of electronically associating the first plurality of biological samples; and
signaling the computer to automatically allocate both the first plurality of biological samples and the second plurality of biological samples to a respective first plurality of wells and a second plurality of wells in a multiwell container.

15. The method of claim 14, further comprising the steps of:
electronically changing the order in which the computer automatically allocates the first plurality of biological samples and the second plurality of biological samples to the first and second plurality of wells by changing the order in which the first plurality of biological samples and the second plurality of biological samples appear on a computer monitor.

16. The method of claim 15, wherein the step of electronically changing the order in which the computer automatically allocates the first plurality of biological samples and the second plurality of biological samples includes the step of moving a data item on the computer monitor indicative of the second plurality of biological samples upward on a sample list displayed on the computer monitor.

17. The method of claim 1, further comprising the steps of:
electronically associating each sample of the first plurality of samples to a corresponding well location in a multiwell container; and
displaying a top view of the multiwell container superimposed with first well indicia indicating each well location to which a sample has been electronically associated.

18. The method of claim 17, further comprising the step of:
displaying the top view of the multiwell container superimposed with second well indicia indicating each well to which a control sample has been electronically associated.

19. The method of claim 17, further comprising the step of:
displaying the top view of the multiwell container superimposed with third well indicia distinguishing wells in the multiwell container containing samples that have been individually named from wells in the multiwell container containing samples that have not been individually named.

20. The method of claim 17, further comprising the step of:
electronically displaying a legend adjacent to the top view explaining what the first well indicia are.

21. The method of claim 17, further comprising the step of:
individually omitting from testing any sample of the first plurality of samples that have been electronically associated to corresponding locations in the multiwell container without changing the location or order of the other samples of the first plurality of samples that have been electronically associated to corresponding locations in the multiwell container.

22. The method of claim 2, further comprising the steps of:
electronically associating each sample of the first plurality of samples to a corresponding location in a multiwell container;
electronically associating each sample of a second plurality of samples associated with the second strain for testing to a corresponding location in the multiwell container;
electronically displaying a top view of the multiwell container superimposed with fourth well indicia distinguishing well locations of the container associated with samples of the first strain, from well locations of the container associated with samples of the second strain.

23. The method of claim 17, further comprising the steps of:
electronically displaying a list of samples of the first plurality of samples associated with a corresponding location in the multiwell container; and
electronically and automatically filling a list of names with an incremented sequence of names for the samples in the list of samples.

24. The method of claim 23, wherein the step of electronically and automatically filling a list of names is based at least upon manual entry of at least two sample names in the list of names.

25. The method of claim 24, further comprising the step of stopping the electronically and automatically filling a list of names when encountering any sample in the list of samples currently having a name.

26. The method of claim 1, wherein the computer is configured to block completion of an order until all of the first plurality of samples have been named, and the names have been transmitted to the computer.

27. The method of claim 1, including the step of:
receiving an automated e-mail notification that the multiwell container has been received at the testing laboratory.

28. The method of claim 27, further comprising the steps of:
placing the first plurality of samples in a multiwell container having indicia; and
sending the multiwell container with samples to the testing laboratory in the package;
wherein the step of receiving the automated e-mail notification includes the step of receiving the automated e-mail notification generated in response to a scan of the indicia at the testing laboratory.

29. The method of claim 1, further comprising the step of:
receiving an automated e-mail notification that biological testing of the first plurality of samples is complete.

30. The method of claim 1, further comprising the steps of:
receiving test results of the biological testing of the first plurality of samples over the Internet, the test results including an identifier of the first strain, an identifier of each probe comprising the first strain, and a list of the first plurality of samples indicating each sample by sample name and by corresponding sample results.

31. The method of claim 30, wherein the sample results for each sample further include an identifier indicating whether the sample was positive or negative for each probe comprising the first strain.

32. The method of claim 30, wherein the test results further include an identifier of a multiwell container in which the first plurality of samples were sent to the testing laboratory.

33. The method of claim 30, wherein the test results further include customer account information and contact information for the testing laboratory.

34. The method of claim 30, wherein the test results further include a date that an order for testing the first plurality of biological samples was placed, a date that the first plurality of biological samples were received at the testing laboratory, and a date that the biological testing of the first plurality of biological samples was completed.

35. The method of claim 30, wherein the test results list each sample of the first plurality of biological samples in the first sample order.

36. The method of claim 30, wherein the test results list each sample of the first plurality of biological samples in an order they were allocated to a multiwell plate.

37. A system for ordering biological tests for biological samples, comprising:
a remote user computer comprising a CPU, and a memory comprising a RAM and a ROM wherein the computer is configured by a plurality of digital instructions to be operable to select a first strain for testing; to select a first plurality of biological samples in a first sample order to be tested for the first strain; and to transmit the first strain associated with the first plurality of biological samples to a second computer configured to receive orders for biological tests.

38. The system of claim 37, wherein the remote user computer is configured by the plurality of digital instructions to be operable to select a second strain for testing; to select a second plurality of biological samples to be tested for the second strain; to transmit over the Internet the second strain associated with the second plurality to the second computer.

39. The system of claim 37, wherein the remote user computer is configured by the plurality of digital instructions to be operable to create the first strain by storing a name of the first strain entered by an operator of the remote user computer; and to store a plurality of probes for selection by the operator of the remote user computer; to associate operator-selected probes of the plurality of probes with the name of the first strain; and to transmit the operator-selected probes to the second computer in association with the name of the first strain.

40. The system of claim 37, wherein the remote user computer is configured by the plurality of digital instructions to be operable to permit an operator of the remote user computer to select a first probe from a plurality of probes, and to associate the first probe with the first strain.

41. The system of claim 37, wherein the first strain includes a plurality of probes, and further wherein the remote user computer is configured by the plurality of digital instructions to permit the operator to first select the plurality of probes by selecting the first strain, and then to deselect at least one of the plurality of probes from the first strain.

42. The system of claim 37, wherein the remote user computer is configured by the plurality of digital instructions to be operable to permit an operator of the remote user computer to associate the first plurality of biological samples with the first strain for testing; and to signal the second computer to automatically allocate the first plurality of biological samples to a corresponding first plurality of wells in a multiwell container in accordance with an electronically predetermined pattern of well filling.

43. The system of claim 38, wherein the remote user computer is configured by the plurality of digital instructions to be operable to permit an operator of the remote user computer to associate the first plurality of biological samples with the first strain for testing; associate the second plurality of biological samples with the second strain for testing after the step of electronically associating the first plurality of biological samples; and to signal the second computer to automatically allocate both the first plurality of biological samples and the second plurality of biological samples to a respective first plurality of wells and a second plurality of wells in a multiwell container.

44. The system of claim 37, wherein the remote user computer is configured by the plurality of digital instructions to be operable to permit an operator of the remote user computer to associate each sample of the first plurality of samples to a corresponding well location in a multiwell container, wherein the plurality of digital instructions further configure a computer display of the remote user computer to display a top view of the multiwell container superimposed with first well indicia indicating each well location to which a sample has been electronically associated.

45. The system of claim 38, wherein the remote user computer is configured by the plurality of digital instructions to be operable to permit an operator of the remote user computer to associate each sample of the first plurality of samples to a corresponding location in a multiwell container and to associate each sample of a second plurality of samples associated with the second strain for testing to a corresponding location in the multiwell container; the plurality of digital instructions further configuring a computer display of said remote user computer to display a top view of the multiwell container superimposed with first well indicia distinguishing well locations of the multiwell container associated with samples of the first strain, from well locations of the multiwell container associated with samples of the second strain.

46. The system of claim 37, wherein the second computer is configured to block completion of an order for biological testing until all of the first plurality of samples have been named by the operator of the remote user computer.

47. The system of claim 37, wherein the remote user computer is configured by the plurality of digital instructions to be operable to permit an operator of the remote user computer to receive at least one of the group comprising e-mail notification that the multiwell container has been received at the testing laboratory, e-mail notification generated in response to a scan of the indicia at the testing laboratory, and e-mail notification that biological testing of the first plurality of samples is complete.

48. The system of claim 37, wherein the remote user computer is configured by the plurality of digital instructions to be operable to permit an operator of the remote user computer to receive test results of the biological testing of the first plurality of samples over the Internet, the test results including an identifier of the first strain, an identifier of each probe comprising the first strain for which the first plurality of samples were tested, and a list of the first plurality of samples indicating each sample by sample name and by corresponding sample results, wherein the sample results for each sample further include an identifier indicating whether the sample was positive or negative for each probe comprising the first strain.
